SEQUENCE LISTING (16 sequences)

SEQ ID NO: 1 - DNA, 975 bp, Artificial Sequence
  Description of Artificial Sequence Synthetic polynucleotide
atgaaatttggattattttttcttaattttataaatagtacaactattcaagaacagtcaatagcaagaatgcaggagattacagagtatgttgataagctaaattttgagcagattcttgtatgtgaaaatcatttttcagataatggtgttgtaggtgctcctttaactgttagtggttttttattaggacttacagaaaaaattaagataggttcattaaatcatgtaattactacacatcatccagttagaatagcagaagaggcttgccttttagatcaactttctgaaggaagatttatattaggttttagtgattgtgaaagaaaagatgagatgcacttttttaatagacctgaacaatatcaacaacaactttttgaagagtgctatgatattataaatgacgcattaactacaggatattgtaatccaaatggagatttttataattttcctaaaatttcagtaaatccacatgcttatactcagaatggtcctagaaagtatgttacagcaacttcttgtcatgtagttgaatgggcagctaagaagggtataccattaatttttaaatgggatgatagtaatgaagtaaaacatgagtatgctaagagatatcaagcaatagctggtgaatatggagttgatcttgcagaaattgatcatcaattaatgatattagttaattattcagaggattctgaaaaagctaaggaagagacaagagcatttataagtgattatattttagctatgcaccctaatgaaaattttgaaaaaaaacttgaggaaataataactgaaaattcagttggtgattatatggagtgcacaactgctgcaaaacttgcaatggaaaaatgtggagctaaaggtattcttttatcttttgaaagtatgtcagattttacacatcagattaatgcaatagatatagtaaatgataatattaagaaatatcatatgtaa

SEQ ID NO: 2 - protein, 360 aa, Artificial Sequence
  Description of Artificial Sequence Synthetic polypeptide
MKFGNFLLTYQPPQFSQTEVMKRLVKLGRISEECGFDTVWLLEHHFTEFGLLGNPYVAAAYLLGATKKLNVGTAAIVLPTAHPVRQLEEVNLLDQMSKGRFRFGICRGLYNKDFRVFGTDMNNSRALMECWYKLIRNGMTEGYMEADNEHIKFHKVKVLPTAYSQGGAPIYVVAESASTTEWAAQHGLPMILSWIINTNEKKAQIELYNEVAQEYGHDIHNIDHCLSYITSVDHDSMKAKEICRNFLGHWYDSYVNATTIFDDSDKTKGYDFNKGQWRDFVLKGHKNTNRRVDYSYEINPVGTPQECIDIIQTDIDATGISNICCGFEANGTVDEIISSMKLFQSDVMPFLKEKQRSLLY

SEQ ID NO: 3 - DNA, 975 bp, Artificial Sequence; identical to SEQ ID NO: 1
  Description of Artificial Sequence Synthetic polynucleotide

SEQ ID NO: 4 - protein, 324 aa, Artificial Sequence
  Description of Artificial Sequence Synthetic polypeptide
MKFGLFFLNFINSTTIQEQSIARMQEITEYVDKLNFEQILVCENHFSDNGVVGAPLTVSGFLLGLTEKIKIGSLNHVITTHHPVRIAEEACLLDQLSEGRFILGFSDCERKDEMHFFNRPEQYQQQLFEECYDIINDALTTGYCNPNGDFYNFPKISVNPHAYTQNGPRKYVTATSCHVVEWAAKKGIPLIFKWDDSNEVKHEYAKRYQAIAGEYGVDLAEIDHQLMILVNYSEDSEKAKEETRAFISDYILAMHPNENFEKKLEEIITENSVGDYMECTTAAKLAMEKCGAKGILLSFESMSDFTHQINAIDIVNDNIKKYHM

SEQ ID NO: 5 - DNA, 1443 bp, Artificial Sequence
  Description of Artificial Sequence Synthetic polynucleotide
atgaataaaaagatatcatttattataaatggaagagttgaaatatttcctgagtcagatgatttagtacaatctataaattttggtgataattctgttcatcttccagtacttaatgattcacaggttaagaatattatagattataatgagaataatgagcttcagcttcataatattataaattttctttatacagtaggacagagatggaagaatgaggagtatagcagaagaagaacttatataagagatcttaagagatatatgggttatagtgaggaaatggcaaaattagaagctaattggatttcaatgatattatgttctaagggaggtttatatgatttagttaaaaatgaattaggaagtagacatattatggatgaatggttacctcaagatgaatcatatataagagcatttccaaaaggtaaaagtgtacatcttttaacaggaaatgttcctttaagtggagtactttcaattttaagagctatacttactaaaaatcagtgcattataaagacatctagtactgatccatttacagcaaatgctttagcacttagttttatagatgttgatcctcatcatccagtaactagatctttaagtgttgtatattggcaacatcaaggtgatatttcacttgctaaagaaataatgcaacatgcagatgttgtagttgcttggggaggtgaagatgcaattaattgggctgtaaagcacgcacctccagatatagatgttatgaaatttggacctaaaaagtctttttgtattatagataatccagtagatttagttagtgctgcaacaggtgctgcacatgatgtatgcttttatgatcagcaggcttgtttttcaactcaaaatatatattatatgggatcacattatgaagaatttaaacttgcattaattgaaaaacttaatttatatgctcatatacttccaaatacaaagaaagattttgatgaaaaggcagcttatagtttagttcagaaagaatgtttatttgcaggacttaaagtagaagttgatgtacatcaaagatggatggttattgaatcaaatgctggtgtagaattaaatcagccacttggaagatgcgtttatttacatcatgtagataatatagagcaaattttaccttatgttagaaagaataaaactcaaacaatatctgtatttccatgggaagcagctttaaagtatagagatcttttagcacttaaaggtgctgaaagaattgttgaggcaggaatgaataatatatttagagtaggtggtgctcatgatggaatgaggcctttacagagacttgttacttatataagtcatgaaagaccaagtcattatacagcaaaagatgtagctgtagagattgagcaaactagatttttagaagaagataagtttttagtatttgttccttaa

SEQ ID NO: 6 - protein, 480 aa, Artificial Sequence
  Description of Artificial Sequence Synthetic polypeptide
MNKKISFIINGRVEIFPESDDLVQSINFGDNSVHLPVLNDSQVKNIIDYNENNELQLHNIINFLYTVGQRWKNEEYSRRRTYIRDLKRYMGYSEEMAKLEANWISMILCSKGGLYDLVKNELGSRHIMDEWLPQDESYIRAFPKGKSVHLLTGNVPLSGVLSILRAILTKNQCIIKTSSTDPFTANALALSFIDVDPHHPVTRSLSVVYWQHQGDISLAKEIMQHADVVVAWGGEDAINWAVKHAPPDIDVMKFGPKKSFCIIDNPVDLVSAATGAAHDVCFYDQQACFSTQNIYYMGSHYEEFKLALIEKLNLYAHILPNTKKDFDEKAAYSLVQKECLFAGLKVEVDVHQRWMVIESNAGVELNQPLGRCVYLHHVDNIEQILPYVRKNKTQTISVFPWEAALKYRDLLALKGAERIVEAGMNNIFRVGGAHDGMRPLQRLVTYISHERPSHYTAKDVAVEIEQTRFLEEDKFLVFVP

SEQ ID NO: 7 - DNA, 924 bp, Artificial Sequence
  Description of Artificial Sequence Synthetic polynucleotide
atggaaaataaaagtagatataagacaatagatcatgttatttgtgtagaggagaatagaaagatacatgtttgggaaactttacctaaagaaaattcaccaaaaagaaaaaatacacttattatagcatctggatttgctagaagaatggatcattttgctggtttagctgaatatttatctcaaaatggatttcatgtaattagatatgattcattacatcatgttggtttaagttcaggaactatagatgaatttacaatgtcaattggtaagcagagtttacttgcagtagttgattggttaaatactagaaaaataaataatcttggaatgttagctagttcattatctgcaagaatagcttatgcaagtctttcagagattaatgtatcttttcttataacagctgttggtgtagttaatttaagatatactttagaaagagcacttggatttgattatcttagccttcctattgatgaattaccagataatcttgattttgagggacataagttaggtgctgaagtatttgcaagagattgctttgattcaggatgggaagatcttacatctactataaatagtatgatgcacttagatattccttttatagcttttacagcaaataatgatgattgggttaaacaagatgaggtaattactcttctttctagtataagaagtcatcagtgtaaaatatattcacttttaggttctagtcatgatcttggagaaaatttagttgtattaagaaatttttatcaatcagttacaaaggctgcaattgctatggataatggttgccttgatatagatgtagatattatagaaccatcttttgagcatttaactattgcagctgttaatgaaagaagaatgaaaatagaaatagagaatcaagtaattagtttaagttaa

SEQ ID NO: 8 - protein, 307 aa, Artificial Sequence
  Description of Artificial Sequence Synthetic polypeptide
MENKSRYKTIDHVICVEENRKIHVWETLPKENSPKRKNTLIIASGFARRMDHFAGLAEYLSQNGFHVIRYDSLHHVGLSSGTIDEFTMSIGKQSLLAVVDWLNTRKINNLGMLASSLSARIAYASLSEINVSFLITAVGVVNLRYTLERALGFDYLSLPIDELPDNLDFEGHKLGAEVFARDCFDSGWEDLTSTINSMMHLDIPFIAFTANNDDWVKQDEVITLLSSIRSHQCKIYSLLGSSHDLGENLVVLRNFYQSVTKAAIAMDNGCLDIDVDIIEPSFEHLTIAAVNERRMKIEIENQVISLS

SEQ ID NO: 9 - DNA, 1113 bp, Artificial Sequence
  Description of Artificial Sequence Synthetic polynucleotide
atgacatcttatgttgataaacaagaaataactgcaagttcagagattgatgatttaatatttagttcagatcctcttgtatggtcttatgatgaacaggaaaagattagaaaaaagttagttcttgatgcttttagacatcattataaacattgtcaagagtatagacattattgccaggcacataaagtagatgataatataacagaaattgatgatataccagtttttcctacttcagtatttaagtttacaagattacttacttcaaatgaaaatgagattgaatcatggtttacaagttcaggaactaatggtttaaaatctcaagttccaagagatagacttagtatagaaagacttttaggatcagtatcttatggtatgaagtatataggaagttggtttgatcatcaaatggagttagttaatcttggtcctgatagatttaatgctcataatatttggtttaaatatgtaatgtcacttgtagaacttttatatccaacaagttttactgtaacagaagagcatatagattttgttcagactttaaatagtcttgaaagaattaaacatcaaggaaaggatatatgtttaattggttcaccttattttatatatcttttatgcagatatatgaaagataagaatatttcttttagtggagataaatcactttatataataactggaggtggatggaaatcttatgaaaaggagagtttaaaaagaaatgattttaatcatcttttatttgatacttttaatctttcaaatattaatcaaataagagatatttttaatcaggtagaattaaatacatgtttttttgaggatgaaatgcaaagaaaacatgttccaccttgggtatatgcaagggctcttgatccagaaactttaaagcctgttccagatggtatgcctggacttatgtcttatatggatgcttcaagtactagttatccagcttttatagtaactgatgatattggtataataagtagagaatatggacaatatcctggagttttagttgagattttaagaagagttaatacaagaaaacagaagggttgtgcactttcattaactgaggcttttggatcttga

SEQ ID NO: 10 - protein, 370 aa, Artificial Sequence
  Description of Artificial Sequence Synthetic polypeptide
MTSYVDKQEITASSEIDDLIFSSDPLVWSYDEQEKIRKKLVLDAFRHHYKHCQEYRHYCQAHKVDDNITEIDDIPVFPTSVFKFTRLLTSNENEIESWFTSSGTNGLKSQVPRDRLSIERLLGSVSYGMKYIGSWFDHQMELVNLGPDRFNAHNIWFKYVMSLVELLYPTSFTVTEEHIDFVQTLNSLERIKHQGKDICLIGSPYFIYLLCRYMKDKNISFSGDKSLYIITGGGWKSYEKESLKRNDFNHLLFDTFNLSNINQIRDIFNQVELNTCFFEDEMQRKHVPPWVYARALDPETLKPVPDGMPGLMSYMDASSTSYPAFIVTDDIGIISREYGQYPGVLVEILRRVNTRKQKGCALSLTEAFGS

SEQ ID NO: 11 - DNA, 7669 bp, Photorhabdus luminescence
gaattctcagactcaaatagaacaggattctaaagacttaagagcagctgtagatcgtgattttagtacgatagagccaacattgagaaattatggggcaacggaagcacaacttgaagacgccagagccaaaatacacaagcttaaccaagaacagaggttatacaaatgacagttaatacagaggcactaataaacagcctaggcaagtcctaccaagaaatttttgatgaagggctaattccttataggaataagccaagtggttctcctggggtgcctaatatttgtattgacatggtgaaagaggggatttttttgtcgtttgaacggaatagtaaaatattaaacgaaattactttaagattgcttagagacgataaagctttgtttatatttccaaatgaattgccatcaccgttgaagcattctatggataggggatgggttagagaaaatttaggtgatctgattaaatcaataccaccgagacaaattttaaaaaggcagtttggttggaaagatctatatcgttttacggatgaaatcagtatgcagatttcttatgatttacgtgaacaggttaattcagtgactttcttgcttacatcagacgtgagttggtaatttaatatatatacccttcatccttcaagttgctgctttgttggctgctttctctcaccccagtcacatagttatctatgctcctggggattcgttcacttgccgccgcgctgcaacttgaaatctattgggtatatgctattggtaattatggaaaattgcctgatttatatataacttaacttgtaaaccagataataatttacatgaatattatcacgtataaaaaaattgcgattcttttaatttgaaatagttcaatttaattgaaactttttattaacaaatcttgttgatgtgaaaattttcgtttgctattttaacagatattgttaaacggagaaggcagcatgttgatgattcactcagccagactgacagttttaagcggaaaattgcagagtatgatcgcattctgataaaggttacaggtcactcgcaaccagaatttcatctttgtatattttgttttgttatttacgttgcagcaagacaaaaatagaagaaacaaatatttatacaacccgtttgcaagagggttaaacagcaatttaagttgaaattgccctattaaatggatggcaaatatgaacaaaaaaatttcattcattattaacggtcgagttgaaatatttcctgaaagtgatgatttagtgcaatccattaattttggtgataatagtgttcatttgccagtattgaatgattctcaagtaaaaaacattattgattataatgaaaataatgaattgcaattgcataacattatcaactttctctatacggtagggcaacgatggaaaaatgaagaatattcaagacgcaggacatatattcgtgatctaaaaagatatatgggatattcagaagaaatggctaagctagaggccaactggatatctatgattttgtgctctaaaggtggcctttatgatcttgtaaaaaatgaacttggttctcgccatattatggatgaatggctacctcaggatgaaagttatattagagcttttccgaaaggaaaatccgtacatctgttgacgggtaatgtgccattatctggtgtgctgtctatattgcgtgcaattttaacaaagaatcaatgcattataaaaacctcatcaactgatccttttaccgctaatgcattagcgctaagttttatcgatgtggaccctcatcatccggtaacgcgttctttgtcagtcgtatattggcaacatcaaggcgatatatcactcgcaaaagagattatgcaacatgcggatgtcgttgttgcttggggaggggaagatgcgattaattgggctgtaaagcatgcaccacccgatattgacgtgatgaagtttggtcctaaaaagagtttttgtattattgataaccctgttgatttagtatccgcagctacaggggcggctcatgatgtttgtttttacgatcagcaagcttgtttttccacccaaaatatatattacatgggaagtcattatgaagagtttaagctagcgttgatagaaaaattgaacttatatgcgcatatattaccaaacaccaaaaaagattttgatgaaaaggcggcctattccttagttcaaaaagaatgtttatttgctggattaaaagtagaggttgatgttcatcagcgctggatggttattgagtcaaatgcgggtgtagaactaaatcaaccacttggcagatgtgtgtatcttcatcacgtcgataatattgagcaaatattgccttatgtgcgaaaaaataaaacgcaaaccatatctgtttttccttgggaggccgcgcttaagtatcgagacttattagcattaaaaggtgcagaaaggattgtagaagcaggaatgaataatatatttcgggttggtggtgctcatgatggaatgagacctttacaacgattggtgacatatatttcccatgaaagaccatcccactatactgctaaagatgttgcggtcgaaatagaacagactcgattcctggaagaagataagttcctggtatttgtcccataataggtaaaagaatatggaaaataaatccagatataaaaccatcgaccatgttatttgtgttgaagaaaatagaaaaattcatgtctgggagacgctgccaaaagaaaatagtccaaagagaaaaaatacccttattattgcgtcgggttttgcccgcaggatggatcattttgccggtctggcagagtatttgtcgcagaatggatttcatgtgatccgctatgattctcttcaccacgttggattgagttcagggacaattgatgaatttacaatgtccataggaaaacagagtttattagcagtggttgattggttaaatacacgaaaaataaataacctcggtatgctggcttcaagcttatctgcgcggatagcttatgcaagtctatctgaaattaatgtctcgtttttaattaccgcagtcggtgtggttaacttaagatatactctcgaaagagctttaggatttgattatctcagcttacctattgatgaattgccagataatttagattttgaaggtcataaattgggtgctgaggtttttgcgagagattgctttgattctggctgggaagatttaacttctacaattaatagtatgatgcatcttgatataccgtttattgcttttactgcaaataatgacgattgggtaaagcaagatgaagttattacattactatcaagcatccgtagtcatcaatgtaagatatattctttactaggaagctcacatgatttgggtgagaacttagtggtcctgcgcaatttttatcaatcggttacgaaagccgctatcgcgatggataatggttgtctggatattgatgtcgatattattgagccgtcattcgaacatttaaccattgcggcagtcaatgaacgccgaatgaaaattgagattgaaaatcaagtgatttcgctgtcttaaaacctataccaatagatttcgagttgcagcgcggcggcaagtgaacgcattcccaggagcatagataactctgtgactggggtgcgtgaaagcagccaacaaagcagcaacttgaaggatgaagggtatattgggatagatagttaactctatcactcaaatagaaatataaggactctctatgaaatttggaaactttttgcttacataccaacccccccaattttctcaaacagaggtaatgaaacggttggttaaattaggtcgcatctctgaggaatgcggttttgataccgtatggttacttgagcatcatttcacggagtttggtttgcttggtaacccttatgtggctgctgcttatttacttggcgcaaccaagaaattgaatgtagggactgcggctattgttctccccaccgctcatccagttcgccagcttgaagaggtgaatttgttggatcaaatgtcaaaaggacgatttcgatttggtatttgtcgggggctttacaataaagattttcgcgtatttggcacagatatgaataacagtcgtgccttaatggagtgttggtataagttgatacgaaatggaatgactgagggatatatggaagctgacaacgaacatattaagttccataaggtaaaagtgctgccgacggcatatagtcaaggtggtgcacctatttatgtcgttgctgaatccgcttccacgactgaatgggccgctcaacatggtttaccgatgattttaagttggattataaatactaacgaaaagaaagcacaaattgagctttataacgaggtcgctcaagaatatggacacgatattcataatatcgaccattgcttatcatatataacatcggtagaccatgactcaatgaaagcgaaagaaatttgccggaattttctggggcattggtatgattcctatgttaatgccacaaccatttttgatgattcagacaaaacaaagggctatgatttcaataaaggacaatggcgcgactttgtcttaaaaggacataaaaatactaatcgtcgcgttgattacagttacgaaatcaatccggtgggaaccccgcaggaatgtattgatataattcaaacagacattgacgccacaggaatatcaaatatttgttgtgggtttgaagctaatggaacagtagatgaaattatctcttccatgaagctcttccagtctgatgtaatgccgtttcttaaagaaaaacaacgttcgctattatattagctaaggaaaatgaaatgaaatttggcttgttcttccttaactttatcaattcaacaactattcaagagcaaagtatagctcgcatgcaggaaataacagaatatgtcgacaaattgaattttgagcagattttggtgtgtgaaaatcatttttcagataatggtgttgtcggcgctcctttgactgtttctggttttttacttggcctaacagaaaaaattaaaattggttcattgaatcatgtcattacaactcatcatcctgtccgcatagcggaagaagcgtgcttattggatcagttaagcgaaggaagatttattttaggatttagtgattgcgagagaaaggatgaaatgcattttttcaatcgccctgaacaataccagcagcaattatttgaagaatgctatgacattattaacgatgctttaacaacaggctattgtaatccaaatggcgatttttataatttccccaaaatatccgtgaatccccatgcttatacgcaaaacgggcctcggaaatatgtaacagcaacaagttgtcatgttgttgagtgggctgctaaaaaaggcattcctctaatctttaagtgggatgattccaatgaagttaaacatgaatatgcgaaaagatatcaagccatagcaggtgaatatggtgttgacctggcagagatagatcatcagttaatgatattggttaactatagtgaagacagtgagaaagctaaagaggaaacgcgtgcatttataagtgattatattcttgcaatgcaccctaatgaaaatttcgaaaagaaacttgaagaaataatcacagaaaactccgtcggagattatatggaatgtacaactgcggctaaattggcaatggagaaatgtggtgcaaaaggtatattattgtcctttgaatcaatgagtgattttacacatcaaataaacgcaattgatattgtcaatgataatattaaaaagtatcacatgtaatataccctatggatttcaaggtgcatcgcgacggcaagggagcgaatccccgggagcatatacccaatagatttcaagttgcagtgcggcggcaagtgaacgcatccccaggagcatagataactatgtgactggggtaagtgaacgcagccaacaaagcagcagcttgaaagatgaagggtatagataacgatgtgaccggggtgcgtgaacgcagccaacaaagaggcaacttgaaagataacgggtataaaagggtatagcagtcactctgccatatcctttaatattagctgccgaggtaaaacaggtatgacttcatatgttgataaacaagaaatcacagcaagttcagaaattgatgatttgattttttcgagtgatccattagtctggtcttacgacgaacaggaaaagattagaaaaaaacttgtgcttgatgcgtttcgtcatcactataaacattgtcaagaataccgtcactactgtcaggcacataaagtagatgacaatattacggaaattgatgatatacctgtattcccaacatcagtgtttaagtttactcgcttattaacttctaatgagaacgaaattgaaagttggtttaccagtagtggcacaaatggcttaaaaagtcaggtaccacgtgacagactaagtattgagaggctcttaggctctgtaagttatggtatgaaatatattggtagttggttcgatcatcaaatggaattggtcaacctgggaccagatagatttaatgctcataatatttggtttaaatatgttatgagcttggtagagttattatatcctacgtcattcaccgtaacagaagaacacatagatttcgttcagacattaaatagtcttgagcgaataaaacatcaagggaaagatatttgtcttattggttcgccatactttatttatttgctctgccgttatatgaaagataaaaatatctcattttctggagataaaagtctttatattataacggggggaggctggaaaagttacgaaaaagaatctttgaagcgtaatgatttcaatcatcttttattcgacactttcaacctcagtaatattaaccagatccgtgatatatttaatcaagttgaactcaacacttgtttctttgaggatgaaatgcaacgtaaacatgttccgccgtgggtatatgcgcgagcacttgatcctgaaacattgaaaccggtacctgatgggatgcctggtttgatgagttatatggatgcatcatcaacgagttatccggcatttattgttaccgatgatatcggaataattagcagagaatatggtcaatatcctggtgtattggttgaaattttacgtcgcgttaatacgaggaaacaaaaaggttgtgctttaagcttaactgaagcatttggtagttgatagtttctttggaaagaggagcagtcaaaggctcatttgttcaatgcttttgcgaaacgttttgtcgaactctaggcgaaggttctcgactttccccgcatcaggggtatatacaagtaaaaaagctcagggggtaaacctgagcttgggatgttgatttttaagtatgagatacatgggcggatttaaataacggagtcagtttggaaatatcaacggtcttttctgctttatcgaggctataagtttcttgcagttttaaccacaaccgcggagagctgccaagtacttgtgacagttttattgccatctctggcgtgactgctgctttacacgatactaaacgttgaaccgtagagggagcaacattcaatgcccgcgctaagttcacgaattc

SEQ ID NO: 12 - DNA, 7803 bp, Artificial Sequence
  Description of Artificial Sequence Synthetic polynucleotide
aattcgaattctcagactcaaatagaacaggattctaaagacttaagagcagctgtagatcgtgattttagtacgatagagccaacattgagaaattatggggcaacggaagcacaacttgaagacgccagagccaaaatacacaagcttaaccaagaacagaggttatacaaatgacagttaatacagaggcactaataaacagcctaggcaagtcctaccaagaaatttttgatgaagggctaattccttataggaataagccaagtggttctcctggggtgcctaatatttgtattgacatggtgaaagaggggatttttttgtcgtttgaacggaatagtaaaatattaaacgaaattactttaagattgcttagagacgataaagctttgtttatatttccaaatgaattgccatcaccgttgaagcattctatggataggggatgggttagagaaaatttaggtgatctgattaaatcaataccaccgagacaaattttaaaaaggcagtttggttggaaagatctatatcgttttacggatgaaatcagtatgcagatttcttatgatttacgtgaacaggttaattcagtgactttcttgcttacatcagacgtgagttggtaatttaatatatatacccttcatccttcaagttgctgctttgttggctgctttctctcaccccagtcacatagttatctatgctcctggggattcgttcacttgccgccgcgctgcaacttgaaatctattgggtatatgctattggtaattatggaaaattgcctgatttatatataacttaacttgtaaaccagataataatttacatgaatattatcacgtataaaaaaattgcgattcttttaatttgaaatagttcaatttaattgaaactttttattaacaaatcttgttgatgtgaaaattttcgtttgctattttaacagatattgttaaacggagaaggcagcatgttgatgattcactcagccagactgacagttttaagcggaaaattgcagagtatgatcgcattctgataaaggttacaggtcactcgcaaccagaatttcatctttgtatattttgttttgttatttacgttgcagcaagacaaaaatagaagaaacaaatatttatacaacccgtttgcaagagggttaaacagcaatttaagttgaaattgccctattaaatggagcatgcggatcctcgactttttaacaaaatatattgataaaaataataggatccgggcccctcgagaggaggatggcaaatatgaataaaaagatatcatttattataaatggaagagttgaaatatttcctgagtcagatgatttagtacaatctataaattttggtgataattctgttcatcttccagtacttaatgattcacaggttaagaatattatagattataatgagaataatgagcttcagcttcataatattataaattttctttatacagtaggacagagatggaagaatgaggagtatagcagaagaagaacttatataagagatcttaagagatatatgggttatagtgaggaaatggcaaaattagaagctaattggatttcaatgatattatgttctaagggaggtttatatgatttagttaaaaatgaattaggaagtagacatattatggatgaatggttacctcaagatgaatcatatataagagcatttccaaaaggtaaaagtgtacatcttttaacaggaaatgttcctttaagtggagtactttcaattttaagagctatacttactaaaaatcagtgcattataaagacatctagtactgatccatttacagcaaatgctttagcacttagttttatagatgttgatcctcatcatccagtaactagatctttaagtgttgtatattggcaacatcaaggtgatatttcacttgctaaagaaataatgcaacatgcagatgttgtagttgcttggggaggtgaagatgcaattaattgggctgtaaagcacgcacctccagatatagatgttatgaaatttggacctaaaaagtctttttgtattatagataatccagtagatttagttagtgctgcaacaggtgctgcacatgatgtatgcttttatgatcagcaggcttgtttttcaactcaaaatatatattatatgggatcacattatgaagaatttaaacttgcattaattgaaaaacttaatttatatgctcatatacttccaaatacaaagaaagattttgatgaaaaggcagcttatagtttagttcagaaagaatgtttatttgcaggacttaaagtagaagttgatgtacatcaaagatggatggttattgaatcaaatgctggtgtagaattaaatcagccacttggaagatgcgtttatttacatcatgtagataatatagagcaaattttaccttatgttagaaagaataaaactcaaacaatatctgtatttccatgggaagcagctttaaagtatagagatcttttagcacttaaaggtgctgaaagaattgttgaggcaggaatgaataatatatttagagtaggtggtgctcatgatggaatgaggcctttacagagacttgttacttatataagtcatgaaagaccaagtcattatacagcaaaagatgtagctgtagagattgagcaaactagatttttagaagaagataagtttttagtatttgttccttaataggaggtaaaagaatatggaaaataaaagtagatataagacaatagatcatgttatttgtgtagaggagaatagaaagatacatgtttgggaaactttacctaaagaaaattcaccaaaaagaaaaaatacacttattatagcatctggatttgctagaagaatggatcattttgctggtttagctgaatatttatctcaaaatggatttcatgtaattagatatgattcattacatcatgttggtttaagttcaggaactatagatgaatttacaatgtcaattggtaagcagagtttacttgcagtagttgattggttaaatactagaaaaataaataatcttggaatgttagctagttcattatctgcaagaatagcttatgcaagtctttcagagattaatgtatcttttcttataacagctgttggtgtagttaatttaagatatactttagaaagagcacttggatttgattatcttagccttcctattgatgaattaccagataatcttgattttgagggacataagttaggtgctgaagtatttgcaagagattgctttgattcaggatgggaagatcttacatctactataaatagtatgatgcacttagatattccttttatagcttttacagcaaataatgatgattgggttaaacaagatgaggtaattactcttctttctagtataagaagtcatcagtgtaaaatatattcacttttaggttctagtcatgatcttggagaaaatttagttgtattaagaaatttttatcaatcagttacaaaggctgcaattgctatggataatggttgccttgatatagatgtagatattatagaaccatcttttgagcatttaactattgcagctgttaatgaaagaagaatgaaaatagaaatagagaatcaagtaattagtttaagttaaaacctataccaatagatttcgagttgcagcgcggcggcaagtgaacgcattcccaggagcatagataactctgtgactggggtgcgtgaaagcagccaacaaagcagcaacttgaaggatgaagggtatattgggatagatagttaactctatcactcaaatagaaatatactgcaggcggccgcaggaggactctctatgaaatttggaaattttttacttacatatcaacctccacagtttagtcaaactgaagttatgaagagattagtaaaacttggtagaatatcagaggaatgtggatttgatacagtttggttacttgaacatcattttactgagtttggtcttttaggaaatccttatgtagcagctgcatatttacttggtgctacaaagaaattaaatgtaggtacagcagctattgttttacctacagcacatcctgttagacagttagaagaagtaaatcttttagatcaaatgtctaaaggtagatttagatttggaatatgcagaggattatataataaggattttagagtttttggtactgatatgaataatagtagggctcttatggagtgttggtataaattaattagaaatggaatgacagaaggttatatggaagcagataatgagcatataaagtttcataaagtaaaagtacttccaactgcttattcacagggaggtgcacctatttatgtagttgctgaatctgcaagtacaactgaatgggctgcacagcatggattaccaatgatactttcatggattataaatacaaatgagaagaaagctcaaatagaattatataatgaagtagcacaagagtatggacatgatattcataatatagatcattgcctttcttatattactagtgttgatcatgattcaatgaaagctaaagaaatatgtagaaattttttaggtcattggtatgattcttatgtaaatgcaacaactatttttgatgatagtgataaaacaaagggatatgattttaataaaggtcagtggagagattttgttcttaaaggacataagaatactaatagaagagtagattattcatatgaaataaatcctgttggaactccacaagagtgtattgatataatacaaactgatattgatgctacaggaatatctaatatttgctgtggatttgaagcaaatggtactgtagatgaaataattagtagtatgaagttatttcagtctgatgttatgccttttcttaaggagaaacaaagaagtttactttattagctaaggaggaaaatgaaatgaaatttggattattttttcttaattttataaatagtacaactattcaagaacagtcaatagcaagaatgcaggagattacagagtatgttgataagctaaattttgagcagattcttgtatgtgaaaatcatttttcagataatggtgttgtaggtgctcctttaactgttagtggttttttattaggacttacagaaaaaattaagataggttcattaaatcatgtaattactacacatcatccagttagaatagcagaagaggcttgccttttagatcaactttctgaaggaagatttatattaggttttagtgattgtgaaagaaaagatgagatgcacttttttaatagacctgaacaatatcaacaacaactttttgaagagtgctatgatattataaatgacgcattaactacaggatattgtaatccaaatggagatttttataattttcctaaaatttcagtaaatccacatgcttatactcagaatggtcctagaaagtatgttacagcaacttcttgtcatgtagttgaatgggcagctaagaagggtataccattaatttttaaatgggatgatagtaatgaagtaaaacatgagtatgctaagagatatcaagcaatagctggtgaatatggagttgatcttgcagaaattgatcatcaattaatgatattagttaattattcagaggattctgaaaaagctaaggaagagacaagagcatttataagtgattatattttagctatgcaccctaatgaaaattttgaaaaaaaacttgaggaaataataactgaaaattcagttggtgattatatggagtgcacaactgctgcaaaacttgcaatggaaaaatgtggagctaaaggtattcttttatcttttgaaagtatgtcagattttacacatcagattaatgcaatagatatagtaaatgataatattaagaaatatcatatgtaatataccctatggatttcaaggtgcatcgcgacggcaagggagcgaatccccgggagcatatacccaatagatttcaagttgcagtgcggcggcaagtgaacgcatccccaggagcatagataactatgtgactggggtaagtgaacgcagccaacaaagcagcagcttgaaagatgaagggtatagataacgatgtgaccggggtgcgtgaacgcagccaacaaagaggcaacttgaaagataacgggtataaaagggtatagcagtcactctgccatatcctttaatattagctgccggctagcaggaggtaaaacaggtatgacatcttatgttgataaacaagaaataactgcaagttcagagattgatgatttaatatttagttcagatcctcttgtatggtcttatgatgaacaggaaaagattagaaaaaagttagttcttgatgcttttagacatcattataaacattgtcaagagtatagacattattgccaggcacataaagtagatgataatataacagaaattgatgatataccagtttttcctacttcagtatttaagtttacaagattacttacttcaaatgaaaatgagattgaatcatggtttacaagttcaggaactaatggtttaaaatctcaagttccaagagatagacttagtatagaaagacttttaggatcagtatcttatggtatgaagtatataggaagttggtttgatcatcaaatggagttagttaatcttggtcctgatagatttaatgctcataatatttggtttaaatatgtaatgtcacttgtagaacttttatatccaacaagttttactgtaacagaagagcatatagattttgttcagactttaaatagtcttgaaagaattaaacatcaaggaaaggatatatgtttaattggttcaccttattttatatatcttttatgcagatatatgaaagataagaatatttcttttagtggagataaatcactttatataataactggaggtggatggaaatcttatgaaaaggagagtttaaaaagaaatgattttaatcatcttttatttgatacttttaatctttcaaatattaatcaaataagagatatttttaatcaggtagaattaaatacatgtttttttgaggatgaaatgcaaagaaaacatgttccaccttgggtatatgcaagggctcttgatccagaaactttaaagcctgttccagatggtatgcctggacttatgtcttatatggatgcttcaagtactagttatccagcttttatagtaactgatgatattggtataataagtagagaatatggacaatatcctggagttttagttgagattttaagaagagttaatacaagaaaacagaagggttgtgcactttcattaactgaggcttttggatcttgaatgcatgtcgactctagagcatgctagtttctttggaaagaggagcagtcaaaggctcatttgttcaatgcttttgcgaaacgttttgtcgaactctaggcgaaggttctcgactttccccgcatcaggggtatatacaagtaaaaaagctcagggggtaaacctgagcttgggatgttgatttttaagtatgagatacatgggcggatttaaataacggagtcagtttggaaatatcaacggtcttttctgctttatcgaggctataagtttcttgcagttttaaccacaaccgcggagagctgccaagtacttgtgacagttttattgccatctctggcgtgactgctgctttacacgatactaaacgttgaaccgtagagggagcaacattcaatgcccgcgctaagttcacgaattc

SEQ ID NO: 13 - DNA, 33 bp, Artificial Sequence
  Description of Artificial Sequence Synthetic primer
cattaggatcctagaatgaagtttcttatgcac

SEQ ID NO: 14 - DNA, 32 bp, Artificial Sequence
  Description of Artificial Sequence Synthetic primer
cattagctcgagaaattttgatacggggtaac

SEQ ID NO: 15 - DNA, 34 bp, Artificial Sequence
  Description of Artificial Sequence Synthetic primer
cattaggatcctaaatgcagaggatgttcttgag

SEQ ID NO: 16 - DNA, 22 bp, Artificial Sequence
  Description of Artificial Sequence Synthetic primer
cactttaacccctcgagtttag

(19) United States
(12) Patent Application Publication
(10) Pub. No.: US 2013/0095509 A1
(43) Pub. Date: Apr. 18, 2013

(54) ENGINEERED LIGHT-EMITTING REPORTER GENES

(71) Applicants: STACY M. BURNS-GUYDISH, CAMPBELL, CA (US); PAMELA REILLY CONTAG, SAN JOSE, CA (US)

(72) Inventors: STACY M. BURNS-GUYDISH, CAMPBELL, CA (US); PAMELA REILLY CONTAG, SAN JOSE, CA (US)

(73) Assignee: COBALT TECHNOLOGIES, INC.

(21) Appl. No.: 13/441,786

(22) Filed: Apr. 6, 2012

Related U.S. Application Data
(63) Continuation of application No. 12/205,845, filed on Sep. 5, 2008, now abandoned.
(60) Provisional application No. 60/970,882, filed on Sep. 7, 2007.

(51) Int. Cl. C12N 15/74

(52) U.S. Cl. CPC C12N 15/74
USPC 435/8; 435/189; 435/252.3; 435/320.1; 536/23.2

(57) ABSTRACT

Compositions and methods are provided for enhanced expression of light emitting reporters. Such reporters are used in methods for monitoring cultures for production of target compounds.

### CROSS-REFERENCE

This application is a continuation application of U.S. Ser. No. 12/205,845, filed on Sep. 5, 2008, which claims the benefit of U.S. Provisional Application No. 60/970,882, filed Sep. 7, 2007, which application is incorporated herein by reference in its entirety.

### BACKGROUND OF THE INVENTION

Light-emitting reporter proteins are commonly used to report various kinds of biological activity. Bioluminescent proteins, such as luciferase, and fluorescent proteins, such as green fluorescent protein, are well known in art. Certain of these proteins have been engineered to produce light of different wavelengths than the naturally occurring protein.

Light-emitting reporters have many applications including construction of biosensors for detection of contaminants, measurement of pollutants, and monitoring of genetically modified organisms released into the environment. Biosensors have also been used as indicators of cellular metabolic activity and for detection of pathogens. In addition, bioluminescent bioreporter organisms that are genetically engineered to produce light when a particular substance is metabolized can be used in other settings. For example, bioluminescent (lux) transcriptional gene fusions may be used to develop light emitting reporter bacterial strains that are able to sense the presence, bioavailability, and biodegradation of organic chemical pollutants such as mercury, naphthalene, toluene, and isopropylbenzene. In general, the lux reporter genes are placed under regulatory control of inducible degradative operons maintained in native or vector plasmids or integrated into the chromosome of the host strain.

### SUMMARY OF THE INVENTION

In general, the invention is directed to products, kits and processes directed to optimized light emitting reporters in microorganisms. In one aspect of the invention it is presented an isolated non-natural nucleic acid molecule comprising a nucleotide sequence encoding a light-emitting reporter, wherein the nucleotide sequence has an A/T content optimized for expression in high-AT microorganism (e.g., Gram positive bacteria).

In various embodiments, the nucleotide sequence encoding a light-emitting reporter comprises an A/T content (over the entire sequence) of between about 62% to about 75%, or about 65% to about 75%. In one embodiment, the A/T content is 69%.

In some embodiments, the light-emitting report is luciferase. In a further embodiment, the sequence encodes a Lux A and/or Lux B polypeptide.

In one embodiment, the light-emitting report is self-contained.

In one embodiment, the nucleotide sequence encodes a Lux A polypeptide having the amino acid sequence of SEQ ID NO: 2. In another embodiment, the nucleotide sequence comprises SEQ ID NO: 1.

In one embodiment, the nucleotide sequence encodes a Lux B polypeptide having the amino acid sequence of SEQ ID NO: 4. In another embodiment, the nucleotide sequence comprises SEQ ID NO: 3.

In some embodiments, the nucleotide sequence encoding a light-emitting reporter is a polycistronic sequence. In a further embodiment, the polycistronic sequence encodes a lux A polypeptide and a lux B polypeptide. In a further embodiment, the sequence encodes a lux A polypeptide, a lux B polypeptide, a lux C polypeptide, a lux D polypeptide and a lux E polypeptide.

In various embodiments, nucleic acid sequence encodes the lux A polypeptide and the lux B polypeptide from Photorhabdus luminescens, Vibrio fischeri, or from a genus of organisms selected from a group consisting of Photorhabdus, Kenorhabdus, and Vibrio.

In various embodiment, the lux polypeptides are from Photorhabdus luminescens.

In some embodiments, the nucleic acid sequence encodes a lux C polypeptide having the amino acid sequence of SEQ ID NO: 6, the lux D polypeptide having the amino acid sequence of SEQ ID NO: 5 and the lux E polypeptide having the amino acid sequence of SEQ ID NO: 10.

In some embodiments, the nucleic acid sequences encode a lux C polypeptide comprises SEQ ID NO: 6, the nucleotide sequence encoding the lux D polypeptide comprises SEQ ID NO: 8 and the nucleotide sequence encoding the lux E polypeptide comprises SEQ ID NO: 10.

In various embodiment, the nucleotide sequence encodes a lux A polypeptide and/or a lux B polypeptide having a non-natural amino acid sequence.

Another aspect of the invention is directed to a recombinant nucleic acid molecule comprising an expression control sequence operatively linked with a coding nucleotide sequence encoding a light-emitting reporter, wherein the coding nucleotide sequence has a high A/T content for expression in a low-GC microorganism. In various embodiments, the A/T content is from about 62% to about 75% or about 65% to about 75%. In one embodiment, the A/T content is 69%.

In a further embodiment, the sequence encodes a Lux A and/or Lux B polypeptide

In one embodiment, the light emitting report is luciferase.

In one embodiment, the recombinant nucleic acid molecule is a plasmid. In another embodiment, the recombinant nucleic acid molecule is a transposon.

In one embodiment, the recombinant nucleic acid molecule comprises an expression control sequence that functions in a gram positive bacterium. For example, in one embodiment, the recombinant nucleic acid molecule comprises a promoter that is functional in Clostridium. In yet further embodiments, the promoter selected is from genes for butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase and butryl CoA transferase.

In another aspect of the invention, the recombinant nucleic acid molecule comprises an operon wherein the coding nucleotide sequence is a polycistronic sequence. For example, in one embodiment, a polycistronic sequence encodes a lux A polypeptide and a lux B polypeptide. In other embodiments, the polycistronic sequence further encodes a lux C polypeptide, a lux D polypeptide and a lux E polypeptide. In one embodiment, the polycistronic sequence encodes ABCDE, CABDE, CADEAB or some other combination of ABCDE. In a further embodiment, the polycistronic sequence further encodes a lux R polypeptide and a lux I polypeptide.

In one embodiment, the polycistronic sequence comprises SEQ ID NO: 12.

In one aspect, the recombinant nucleic acid molecule comprises an expression control sequence comprising a Shine-Dalgarno sequence (AGGAGG) operatively linked with each cistron.

In one embodiment, the recombinant nucleic acid molecule comprises a first restriction sequence upstream of the expression control sequence, a second restriction sequence between a promoter of the expression control sequence and the coding nucleotide sequence and a third restriction sequence downstream of the coding nucleotide sequence. In another embodiment, a first restriction sequence and a second restriction sequence upstream and downstream, respectively, of the sequence encoding the lux A polypeptide and the lux B polypeptide.

In another aspect of the invention, a recombinant cell comprising a recombinant nucleic acid molecule comprising an expression control sequence operatively linked with a coding nucleotide sequence encoding a light-emitting reporter, wherein the coding nucleotide sequence has a high A/T content. In various embodiments, the AT content is from between about 62% to about 75%, about 65% to about 75%, or about 62% to about 75%. In one embodiment, the A/T content is 69%.

In some embodiments, the cell is a Clostridium cell, such as Clostridium is C. acetobutylicum, C. perfringens, C. saccharobutylicum, C. puniceum, C. saccharoperobutylicum or C. beijerinckii.

In one embodiment, the cell is another bacteria with an AT rich DNA

In one embodiment, the recombinant cell comprises the recombinant nucleic acid which is not integrated into the cell genome, while in another embodiment, the recombinant nucleic acid is integrated into the cell genome.

In various embodiments, a recombinant cell may comprise a plurality of different recombinant nucleic acid molecules, wherein the different recombinant nucleic acid molecules comprise different expression control sequences and different coding nucleotide sequences encoding light-emitting reporters that report light of different wavelengths.

In another aspect of the invention, an isolated polypeptide comprising a light-emitting reporter, wherein the polypeptide is encoded by a nucleotide sequence having a high A/T content for expression in a low-GC microorganism. In various embodiments, the AT content is from between about 62% to about 75%, about 65% to about 75%, or about 62% to about 75%. In one embodiment, the A/T content is 69%.

In one embodiment, the light-emitting reporter is luciferase. In a further embodiment, the light-emitting report is self-contained.

In some embodiments, the light-emitting reporter further comprises a Lux A polypeptide and/or a Lux B polypeptide.

In one embodiment, the polypeptide comprises the amino acid sequence of SEQ ID NO: 2.

In another embodiment, the polypeptide is encoded by the nucleotide sequence of SEQ ID NO: 1.

In one embodiment, the polypeptide comprises the amino acid sequence of SEQ ID NO: 4.

In another embodiment, the polypeptide is encoded by the nucleotide sequence comprises SEQ ID NO: 3.

In one embodiment, the polypeptide is encoded by a polycistronic sequence encoding luciferase.

In one embodiment, the polypeptide comprises Lux A and Lux B. In further embodiment, the lux A polypeptide and the lux B polypeptide are from Photorhabdus luminescens, Vibrio fischeri or from a genus of organisms selected from a group consisting of Photorhabdus, Kenorhabdus, and Vibrio.

In further embodiment, the polycistronic sequence encodes a lux A polypeptide, a lux B polypeptide, a lux C polypeptide, a lux D polypeptide and a lux E polypeptide.

In one embodiment, the lux polypeptides are from Photorhabdus luminescens.

Another aspect of the invention is directed to a method comprising:

(a) culturing a recombinant cell comprising a recombinant nucleic acid molecule comprising an expression control sequence operatively linked with a coding nucleotide sequence encoding a light-emitting reporter, wherein the coding nucleotide sequence has an A/T content from between about 62% to about 75%, about 65% to about 75%, or about 62% to about 75%; and

(b) measuring the light emitted from the reporter in the culture.

In one embodiment, the light-emitting reporter is self-contained. In another embodiment, the AT content is about 69%.

In various embodiments, the cell is Clostridium and the expression control sequence comprises a Clostridium promoter.

In one embodiment, the expression control sequence is from a low-GC bacteria.

In another embodiment, the light-emitting reporter is from Photorhabdus luminescens.

In one aspect of the invention comprises a method for identifying and/or optimizing fermentation culture conditions comprising: culturing a plurality of cultures, wherein the bacteria are the same, wherein the culture conditions are different, and wherein one culture condition serves as a control condition; monitoring the expression of a light emitting reporter in said bacteria in said cultures, wherein said light emitting reporter is encoded by a nucleic acid sequence comprising total A/T content of about 62% to about 75%; and wherein said bacteria is low-GC bacteria; and identifying said cultures that have a higher or lower expression of the light emitting reporter compared to a control culture.

In one embodiment, the cultures with a higher expression of the light emitting report compared to the control culture indicate culture conditions that will result in higher productivity than a control culture condition. In another embodiment, the culture conditions vary be nutrient, vitamin, mineral, salt, or cofactor composition. In a further embodiment, the culture conditions vary by a physical parameter selected from temperature, pH, oxygen partial pressure, osmotic pressure, or dilution rate of said culture.

In one aspect of the invention a method is provided for identifying mutants with higher productivity comprising: mutagenizing a plurality of bacteria that express a recombinant nucleic acid molecule comprising an expression control sequence operatively linked with a coding nucleotide sequence encoding a light-emitting reporter, wherein the coding nucleotide sequence has an A/T content between about 62% and about 75%; isolating pure cultures derived from individual mutants; culturing the pure cultures of mutants; measuring the light emitted from the reporter in the cultures; and selecting mutants that have a higher emission of light than an unmutagenized parent strain.

In another aspect of the invention, a kit is provided comprising: a first container containing a first nucleic acid molecule comprising an expression control sequence; and a second container containing a second nucleic acid molecule comprising a coding nucleotide sequence encoding a light-emitting reporter, wherein the coding nucleotide sequence has an A/T content between about 62% to about 75%, about 65% to about 75%, or about 62% to about 75%; wherein the first and second nucleic acid molecules comprise compatible restriction sequences which, when the first and second nucleic acid molecules are ligated together, put the expression control sequence in operative linkage with the coding nucleotide sequence and create a restriction sequence. In one embodiment, the AT content is 69%.

In another aspect of the invention a kit is provided comprising: a Clostridium cell; and recombinant nucleic acid molecule comprising an expression control sequence operatively linked with a coding nucleotide sequence encoding a light-emitting reporter, wherein the coding nucleotide sequence has an A/T content between about 62% to about 75%, about 65% to about 75%. In one embodiment, the AT content is about 69%.

### INCORPORATION BY REFERENCE

All publications and patent applications mentioned in this specification are herein incorporated by reference to the same extent as if each individual publication or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a schematic representation of an expression construct comprising lux CDABE that was optimized for expression in a low DNA G+C content bacterium such as a Clostridium species. Ribosome binding sites (asterisks) were inserted upstream of each gene.

FIG. 2 illustrates a pathway of light production in the bacterial bioluminescence system (modified from Meighen 1988).

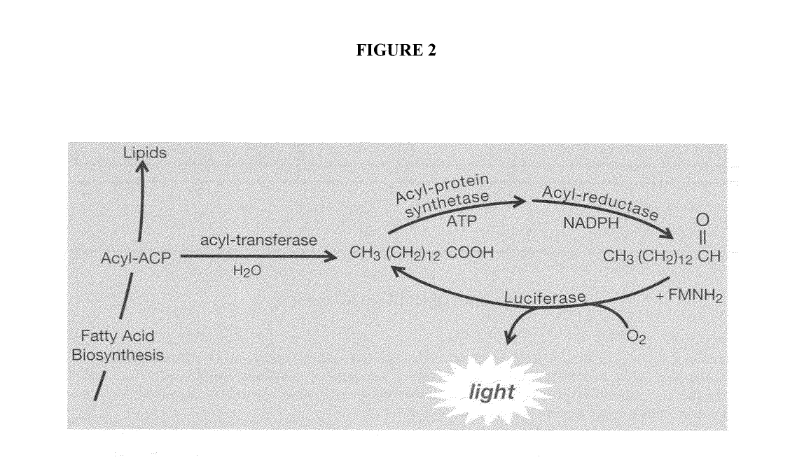

FIG. 3 demonstrates the functioning of high A/T optimized lux* cassettes in E. coli. Relative luminescence was measured using a luminescence plate reader. E. coli K12 were transformed with the pUC19-luxABE or pUC19-thl-luxCDABE. Nontransformed E. coli K12 served as control. pUC19-thl-luxCDABE produced approximately 10⁶ RLU/10 sec without the addition of substrate. The optimized partial cassette, pUC19-luxABE produced luminescence at approximately 10⁵ RLU/10 sec and 6×10³ RLU/10 sec, with and without substrate, respectively.

FIG. 4 illustrates the expression of optimized pJIR418-lux* cassettes in Clostridium beijerinckii. Strain Co-0124 was electroporated with an optimized luxCDABE* cassette on a shuttle vector without (A) a functioning Clostridium promoter (pJIR418-lux*) or with (B) a functioning Clostridium promoter (bdhB, inducible promoter, pJIR418-bdhB-lux*). Cells were plated on agar plates, incubated and once colonies developed the plates were briefly exposed to oxygen before imaging. No light was produced by cells transformed with the promoterless luxCDABE* cassette while cells transformed with the bdhB-luxCDABE* cassette produced light. Bioluminescence was measured with an In Vivo Imaging System (IVIS, Caliper Life Science, Hopkinton, Mass.) and was displayed in photons/sec as shown by the rainbow scale. (C) Comparison of transformants grown in liquid culture are shown with and without a functioning promoter demonstrating 3-logs difference in luminescence.

FIG. 5 demonstrates the expression of an optimized lux cassette (pJIR418-bdhB-lux*) in a different Clostridium species, C. acetobutylicum.

FIG. 6 is a comparison of bioluminescence and butanol production in C. beijerinckii expressing optimized pJIR418-lux* cassettes. Bioluminescence total flux (photons/sec) was detected with an IVIS and butanol formation was determined by HPLC from small samples of the culture over time. (A) The Clostridium strain Co-0124 with the promoterless lux construct (pJIR418-lux*) demonstrated butanol formation but no significant light production over background levels (˜104 p/s). (B) By comparison, Co-0124 with the bdhB-lux*(pJIR418-bdhB-lux*) construct had a 100-fold increase in light production during butanol formation followed by a dramatic decrease in light production when butanol formation ceased.

FIG. 7 demonstrates that with the constitutive promoter thiolase (thl) (pJIR418-thl-lux*) bioluminescence correlates with the growth rate of the culture. The increase in bioluminescence precedes, but mirrors the increase in OD. Bioluminescense increases rapidly during the exponential phase of growth, but peaks prior to the peak in OD, before gradual declining during the platue phase of cell growth. Butanol, on the other hand, continues to accumulate.

FIG. 8 illustrates that lux expression is a more sensitive and direct indicator of butanol formation compared to monitoring butanol in the offgas by mass spectrometry, monitoring butanol in the fermentation broth by HPLC, or by measuring the increase in cell mass by spectrometry (optical density).

FIG. 9 illustrates that the detection of bioluminescence precedes the detection of butanol formation in batch culture and correlates well with overall butanol productivity. Two different batch fermentations of the C. beijerinckii sensor strain, Co-5878 are shown.

FIG. 10 illustrates the monitoring of bioluminescence in a continuous fermentation of the C. beijerinckii sensor strain Co-5878. Changes in bioluminescence precede the fluctuations in the butanol production rate.

FIG. 11 demonstrates the real-time changes in bioluminescence for different culture conditions of C. beijerinckii strain Co-5878 utilizing an IVIS and are graphed as total flux (p/s) over time. Several candidate conditions increased the initial peak in light production and/or had a prolonged production of light over time. Two culture conditions, the addition of vitamins and phosphate limitation were chosen as fermentation conditions to be analysized for their effect on butanol productivity in 15 L fermentations. These two fermentation conditions increased butanol productivity compared to a control fermentation (data shown in Table 2).

### DETAILED DESCRIPTION OF THE INVENTION

In certain embodiments the present invention provides genes encoding light-emitting reporter proteins, such as bioluminescent reporters or fluorescent reporters (in particular the Lux proteins of luciferase) that are genetically modified to have nucleotide sequences that are A/T rich, that is, to have A/T content of at least 62%. These genes are useful, among other things, for expression and activity in cells (e.g., Clostridium) that have a preference for A/T rich genes. To make such genes codons are designed to replace G or C with A or T, in particular positions that do not change the amino acid encoded at that codon. This is accomplished, of course, by taking advantage of the degeneracy of the genetic code so as to replace codons that include C or G at degenerate positions with A or T. In certain cases, such as lux, a reporter construct is part of a larger operon containing several cistrons. In this case, the entire coding sequence of the operon can be engineered to have A/T rich content. These engineered genes then can be operatively linked with expression control sequences, such as promoters and/or ribosome binding sites, that are compatible with the intended host organism.

This invention further provides compositions and methods designed to monitor cell growth and cell fitness. Furthermore, the compositions and methods provide for real time monitoring and analysis of various pathways in cellular metabolism (e.g., solventogenesis and acidogenesis) utilizing a reporter. In various embodiments of the invention, the reporter is a light emitting reporter optimized for use in the desired host cell. For example, in various embodiments a light emitting reporter is engineered to express in an obligate or strict anaerobe bacterium. Furthermore, by selecting the appropriate promoter, such expression can be linked to both gene and pathway expression.

As such, the monitoring of the reporter expression may be used to monitor the physiological state of the culture. In some embodiments, a detected signal using such a system is utilized as a control signal for hardware and software that can regulate the fermentation process (e.g., microbial batch, fed-batch or continuous culture).

The practice of the present invention will employ, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Ausubel, F. M., et al., Current Protocols in Molecular Biology, John Wiley and Sons, Inc., Media, Pa. (1995.); Sambrook, J., et al., Molecular Cloning: A Laboratory Manual, Third Edition, Cold Spring Harbor Laboratory (Cold Spring Harbor, N.Y.) (2001).)

### DEFINITIONS

As used herein, the term “vector” refers to a polynucleotide construct, typically a plasmid or a virus, used to transmit genetic material to a host cell. Preferably, the term “vector” as used herein refers to an agent such as a plasmid, and even more preferably to a circular plasmid. A vector as used herein may be composed of either DNA or RNA. Preferably, a vector as used herein is composed of DNA.

As used herein, the term “episomally replicating vector” or “episomal vector” refers to a vector which is typically and very preferably not integrated into the genome of the host cell, but exists in parallel. An episomally replicating vector, as used herein, is replicated during the cell division and in the course of this replication the vector copies are included in each daughter cell.

“Operatively linked” or “operably linked” refers to a functional arrangement of elements wherein the activity of one element (e.g., a promoter) results on an action on the other element (e.g., a nucleotide sequence). Thus, a given promoter that is operably linked to a coding sequence (e.g., a reporter gene) is capable of effecting the expression of the coding sequence when the proper enzymes are present. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Substantially pure” or “isolated” means an object species is the predominant species present (i.e., on a molar basis, more abundant than any other individual macromolecular species in the composition), and a substantially purified fraction is a composition wherein the object species comprises at least about 50% (on a molar basis) of all macromolecular species present. Generally, a substantially pure composition means that about 80% to 90% or more of the macromolecular species present in the composition is the purified species of interest. The object species is purified to essential homogeneity (contaminant species cannot be detected in the composition by conventional detection methods) if the composition consists essentially of a single macromolecular species. Solvent species, small molecules (<500 Daltons), stabilizers (e.g., BSA), and elemental ion species are not considered macromolecular species for purposes of this definition.

The terms “non-natural” or “non-naturally occurring” as used herein refer to a compound or composition that does not occur in nature or is engineered using recombinant technology to modify the compound or composition to something different than something occurring in nature (e.g., nucleic acid molecule that is codon optimized as compared to what is present in nature).

An “expression cassette” comprises any nucleic acid construct which contains a promoter operatively linked with polynucleotide gene(s) or sequence(s).

As used herein, the term “gene of interest” refers to a nucleic acid sequence comprising the coding sequence for the gene of interest which can be either spaced by introns or which is a cDNA encoding the open reading frame. Typically and preferably, the term “gene of interest”, as used herein, refers to a nucleic acid sequence further comprising a promoter, preferably a promoter that activates the gene of interest, and even more preferably, to a nucleic acid sequence further comprising a promoter and a polyadenylation signal sequence. This nucleic acid sequence may again further comprise an enhancer. For example, gene(s) of interest include those which encode lux protein or luciferase optimized for expression in a host cell as described herein.

“Recombinant” as used herein to describe a nucleic acid molecule, means a polynucleotide of genomic, cDNA, semisynthetic, or synthetic origin which, by virtue of its origin or manipulation: (1) is not associated with all or a portion of the polynucleotide with which it is associated in nature; and/or (2) is linked to a polynucleotide other than that to which it is linked in nature. The term “recombinant” as used with respect to a protein or polypeptide means a polypeptide produced by expression of a recombinant polynucleotide. “Recombinant host cells,” “host cells,” “cells,” “cultures” and other such terms denoting prokaryotic microorganisms cultured as unicellular entities, are used interchangeably, and refer to cells which can be, or have been, used as recipients for recombinant vectors or other transfer DNA, and include the progeny of the original cell which has been transformed. It is understood that the progeny of a single parental cell may not necessarily be completely identical in morphology or in genomic or total DNA complement to the original parent, due to accidental or deliberate mutation. For example, recombinant cells of the invention include Gram positive low content G+C DNA bacteria which are engineered to include expression-optimized genes encoding light emitting reporters.

In various embodiments, compositions of the invention include sequences having sequence identity to a certain level as compared to sequences disclosed herein. Techniques for determining nucleic acid and amino acid “sequence identity” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra

Two nucleic acid fragments are considered to “selectively hybridize” as described herein. The degree of sequence identity between two nucleic acid molecules affects the efficiency and strength of hybridization events between such molecules. A partially identical nucleic acid sequence will at least partially inhibit a completely identical sequence from hybridizing to a target molecule Inhibition of hybridization of the completely identical sequence can be assessed using hybridization assays that are well known in the art (e.g., Southern blot, Northern blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a target nucleic acid sequence, and then by selection of appropriate conditions the probe and the target sequence “selectively hybridize,” or bind, to each other to form a hybrid molecule. A nucleic acid molecule that is capable of hybridizing selectively to a target sequence under “moderately stringent” conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90%-95% with the sequence of the selected nucleic acid probe. Hybridization conditions useful for probe/target hybridization where the probe and target have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Therefore, in various embodiments of the invention, sequences having from about 70 to about 99, about 80 to about 99 and 90 to about 100% identity are contemplated for use in compositions and methods of the invention. In some embodiments, such sequences function similarly to the disclosed sequences but have sequence identity of from 70% to 99%, including 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or about 100%.

With respect to stringency conditions for hybridization, it is well known in the art that numerous equivalent conditions can be employed to establish a particular stringency by varying, for example, the following factors: the length and nature of probe and target sequences, base composition of the various sequences, concentrations of salts and other hybridization solution components, the presence or absence of blocking agents in the hybridization solutions (e.g., formamide, dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.)

“Encoded by” refers to a nucleic acid sequence which codes for a polypeptide sequence, wherein the polypeptide sequence or a portion thereof contains an amino acid sequence of at least 3 to 5 amino acids, or at least 8 to 10 amino acids, or at least 15 to 20 amino acids from a polypeptide encoded by the nucleic acid sequence. Also encompassed are polypeptide sequences which are immunologically identifiable with a polypeptide encoded by the sequence.

The term “Gram-positive” is a taxonomic feature referring to bacteria which resist decolorization with any standard Gram-staining dyes. In contrast, Gram-negative bacteria are easily decolorized with certain organic solvents such as ethanol or acetone. The ability of bacteria to retain or resist staining generally reflects the structure of the cell wall and it has been suggested that Gram-positive bacteria have more extensive peptidoglycan crosslinking and less permeable cells walls than their Gram-negative counterparts. Non-limiting examples of Gram-positive bacteria include: Stapholococcus, Streptococcus, certain Bacillus, Anthrax, Mycobacterium, etc.

In various embodiments, a light emitting reporter is optimized for expression in Gram positive bacteria. For example, certain Gram positive bacteria (e.g., Clostridium acetobutylicum and other gram positive anaerobes) do not optimally express bacterial luciferase due to disparate G+C content, incompatible ribosome binding sites and possibly other incompatible transcriptional regulatory elements. In addition bacterial luciferase requires oxygen as a substrate.

“Light-emitting” is defined as capable of generating light through a chemical reaction or through the absorption of radiation.

“Light” is defined herein, unless stated otherwise, as electromagnetic radiation having a wavelength of between about 300 nm and about 1100 nm.

“Visible light” is defined herein, unless stated otherwise, as electromagnetic radiation having a wavelength of between about 400 nm and about 750 nm.

“Light-emitting protein” or “light-emitting reporter” is defined as a protein or polypeptide capable of generating light through a chemical reaction (e.g., bioluminescence, as generated by luciferase) or through the absorption of radiation (e.g., fluorescence, as generated by Green Fluorescent Protein).

“Luciferase,” unless stated otherwise, includes prokaryotic and eukaryotic luciferases, as well as variants possessing varied or altered optical properties, such as luciferases that produce different colors of light (e.g., Kajiyama, N., and Nakano, E., (1991) Protein Engineering 4(6):691-693. “Lux” refers to prokaryotic genes associated with luciferase and photon emission. “Luc” refers to eukaryotic genes associated with luciferase and photon emission. Luciferase is a low molecular weight oxidoreductase which catalyzes the dehydrogenation of luciferin or other substrate in the presence of oxygen, ATP and magnesium ions. During this process, about 96% of the energy released appears as visible light. Luciferase is a well known real time reporter protein and can be expressed in most Gram negative aerobic bacteria and some Gram positive aerobes.

Luciferases are oxygenases that act on a substrate which, through an enzyme catalyzed reaction in the presence of molecular oxygen and ATP, transform the substrate into an excited state. Due to the physical principal of conservation of energy, when the substrate returns to a lower energy state, it releases energy in the form of light (a phenomena called bioluminescence). The color or wavelength of the emitted light in a reaction is a unique characteristic of the excited molecule, and is independent from its source of excitation. An essential condition for bioluminescence is the use of molecular oxygen, either bound or free in the presence of a luciferase. Since luciferases are proteins, their function can be altered through a process called mutagenesis.

Bacterial luciferase (“lux”) is typically made up of two subunits (α and β) encoded by two different genes (luxA and luxB) on the lux operon. Three other genes on the operon (lux C, lux D and luxE) encode the enzymes required for biosynthesis of the aldehyde substrate. Bacterial lux is present in certain bioluminescent Gram-negative bacteria (e.g., Photorhabdus luminescens) and is ordered CDABE.

In some embodiments, the function of any light-emitting reporter can be modified by addition of another protein or substrate which functions to shift the detectable wavelength. For example, a second fluorescent protein can be expressed whereby concomitant expression of an ‘optimized’ reporter results in a wavelength shift, thus providing a unique reporter or detectable signal. As such, in various embodiments of the invention, a primary ‘optimized’ reporter (e.g., SEQ ID NO: 12) can be co-expressed with a second protein (e.g., introduced via second expression construct which can be integrated into the host genome, expressed from a different vector or the same vector). Such secondary reporter proteins and substrates are further described herein and known in the art.

### 1. Nucleic Acid Constructs

1.1. Sequence Optimization

In one aspect of the invention, sequences encoding a light-emitting reporter are optimized for expression in a host cell. For example, sequences encoding a light-emitting reporter can be optimized for expression in Gram positive anaerobes of high A/T content. Thus one sub-aspect of the invention is directed to altered sequences or codon usage manipulation for expression of the altered sequence in a Gram positive bacteria. In various embodiments, sequences are codon optimized to comprise high A/T content for expression in low-GC bacteria. In further embodiments, such low-GC bacteria are obligate or strict anaerobe Gram positive bacteria.

In various embodiments, nucleic acid sequences encoding a light emitting reporter are altered to comprise A/T content of from about 62% to about 75%, about 62% to about 65%, 62% to 70%, 65% to 75% or 70% to 75% of the total sequence based on codon degeneracy. Thus in various embodiments, A/T content is about 62%, 63%, 64%, 65%, 66%, 67%, 68%, 69%, 70%, 71%, 72%, 73%, 74% or 75%.

In some embodiments, lux genes are optimized to comprise A/T content from about 62% to about 75%, about 62% to about 65%, 62% to 70%, 65% to 75% or 70% to 75% of the total sequence based on codon degeneracy. Thus in various embodiments, A/T content is about 62%, 63%, 64%, 65%, 66%, 67%, 68%, 69%, 70%, 71%, 72%, 73%, 74% or 75%.

In a further embodiment, a lux operon sequence is optimized for expression in Gram positive anaerobes of high A/T (i.e., low-GC) content, with a specified ribosome binding site and an altered substrate binding site. Therefore, in one embodiment, a lux operon can be modified as to A/T content (for illustrative purposes, A/T optimization=“X”). In another embodiment, the lux operon can be modified as to A/T content and as to ribosome binding sites (particular to the desired host) (for illustrative purposes, ribosome binding site modification=“Y”). In yet another embodiment, a lux operon is modified to alter the LuxA catalytic site for substrate binding (for illustrative purposes, substrate binding site modification=“Z”). Therefore, in various embodiments, a nucleic acid construct of the invention comprises X, XY, XYZ or XZ. In yet a further embodiment, any of the preceding can be modified by a promoter or expression control sequence obtained from the particular host in which the nucleic acid construct (X, XY, XYZ or XZ) is integrated or provided via an episomal vector.

As such, luciferase reporters can be used to track gene expression in bacteria (e.g., Gram positive anaerobe). For example, Clostridial codon usage for a low-G/C bacteria (e.g., GC content is less than about 40%) can be optimized for the enhanced expression. In one embodiment, lux genes are optimized and ribosome binding sites are provided upstream of each lux gene to allow expression in a desired bacterial host. In a further embodiment, an expression construct is provided to a host cell episomally or integrated, wherein the expression construct comprises the sequence of SEQ ID NO: 12.

In one embodiment, the expression construct comprises the sequence of SEQ ID NO: 1 and SEQ ID NO: 3. In yet further embodiments, the expression construct comprises the sequences of SEQ ID NO: 1, 3, 5, 7 and 9. It should be noted that where sequences are illustrated with a stop codon, any equivalent stop codon can be used (e.g., UAG (“amber”), UAA (“ochre”), and UGA (“opal” or “umber”). Furthermore, if a sequence encoding a protein is listed without a stop codon, it will be evident to one of ordinary skill, that any stop codon can be used here as well.

As noted above, the range of wavelengths which can be used in various methods (e.g., monitoring) can be extended and expanded by co-expressing various additional reporters and/or providing additional substrates to effect a shift in wavelength. In other words, the wavelength that is detectable is different than if the optimized light emitting reporter were expressed alone. As such, in various further embodiments, multiple different reporters (e.g., detectable at different wavelengths (color) allow for multiparameter studies.

In yet other embodiments, wavelengths are altered by altering pH in the cell culture. For example, a light emitting reporter may function better at one pH (e.g, low pH) versus a different light emitting reporter which functions at a higher pH. As such, depending on the host cell and culture conditions a reporter is selected as desired. Furthermore, certain host cells (e.g., C. acetobutylicum) have different growth phases that either prefer a certain pH range or will change the culture pH range by the secretion of organic acids. For example. C. acetobutylicum growing in the acidogenic phase may lower culture medium pH (e.g., pH of 4 to 5.5).

In another aspect of the invention, the optimization of a sequence encoding a light emitting reporter containing an AT content of about 62 to about 75% provides a protein that functions as an oxygenase or oxygen scavenging protein. Thus, in one embodiment, where such optimized sequences are expressed in a microorganism (e.g., in an oligate or strictly anaerobic bacterium), such microorganisms are able to grow in a low oxygen environment or under partial oxygen pressure, because of said optimization.

1.2. Light Producing Molecules

The light producing molecules useful in the practice of the present invention may take any of a variety of forms, depending on the application. They share the characteristic that they are luminescent, that is, that they emit electromagnetic radiation in ultraviolet (UV), visible and/or infra-red (IR) from atoms or molecules as a result of the transition of an electronically excited state to a lower energy state, usually the ground state. Examples of light producing molecules include photoluminescent molecules, such as fluorescent molecules, chemiluminescent compounds, phosphorescent compounds, and bioluminescent molecules.

In certain embodiments, the light-emitting reporter is self-contained. As used herein, a light-emitting reporter is “self-contained” if it produces light without the addition of exogenous organic substrate. Thus, for example, fluorescent reporters are “self-contained.” The lux operon, which produces microbial luciferase, is also a self-contained reporter in that it contains enzymes to produce the necessary substrate. By contrast, the luc gene, which produces a eucaryotic luciferase, requires the addition of a substrate such as luciferin and frequently ATP in order for there to be bioluminescence. Therefore, it is not self-contained. Self-contained reporters provide certain advantages in the methods of this invention because the addition of exogenous substrate can be expensive and introduce inefficiencies into monitoring and regulating the state of the culture.

1.2.1. Bioluminescent Proteins

Bioluminescent molecules are distinguished from fluorescent molecules in that they do not require the input of radiative energy to emit light. Rather, bioluminescent molecules utilize chemical energy, such as ATP, to produce light. An advantage of bioluminescent molecules, as opposed to fluorescent molecules, is that there is less background signal in the environment compared to background fluorescence. The only light detected in a dark environment is light that is produced by the exogenous bioluminescent molecule. In contrast, the light used to excite a fluorescent molecule often results in background fluorescence through the auto fluorescence of non-target compounds in the environment that interferes with signal measurement.

Several types of bioluminescent molecules are known. They include the luciferase family (de Wet, J. R, et al., Firefly luciferase gene: structure and expression in mammalian cells. Mol. Cell. Biol. 7:725-737, 1987) and the aequorin family (Prasher, et al. Cloning and expression of the cDNA coding for Aequorin, a bioluminescent calcium-binding protein. Biochem Biophys Res Commun 126: 1259-1268, 1985). Members of the luciferase family have been identified in a variety of prokaryotic and eukaryotic organisms. Prokaryotic luciferase is encoded by two subunits (luxAB) of a five gene complex that is termed the lux operon (luxCDABE). The remaining three genes comprise the luxCDE subunits and code for the fatty acid reductase responsible for the biosynthesis of the aldehyde substrate used by luciferase for the luminescent reaction.

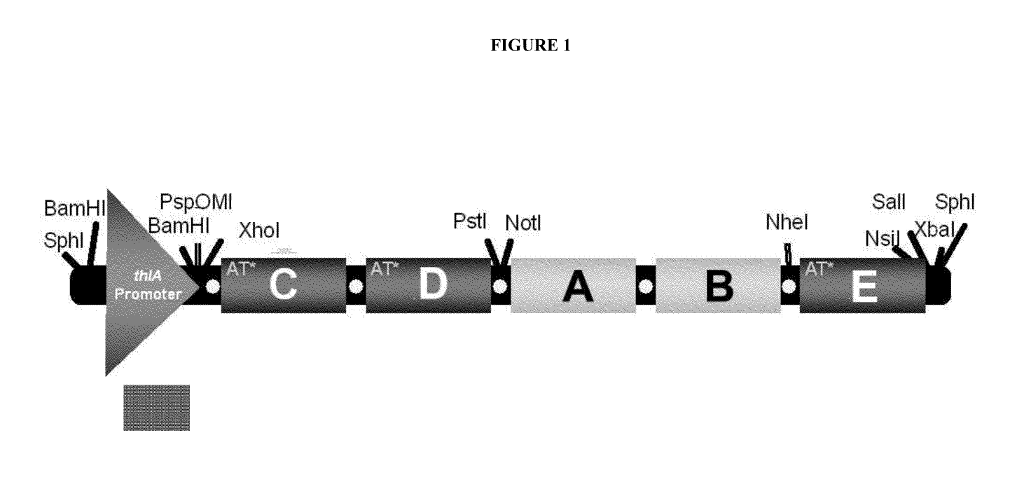

The synthesis of light in naturally occurring bioluminescent bacteria is encoded by five essential genes. These genes are clustered in an operon (luxCDABE; FIG. 1) that can be moved into non-bioluminescent bacteria to produce a bioluminescent phenotype. Since all identified species of naturally occurring marine and terrestrial bioluminescent bacteria are Gram-negative however, the transformation of Gram-positive bacteria to bioluminescent phenotype has been limited, due in part to the differing genetics of these two bacterial groups. The present invention solves this problem in one aspect by reengineering the entire Photorhabdus luminescens lux operon (SEQ ID NO: 11) to introduce A/T content necessary to express in high A/T Gram positive bacterium (SEQ ID NO: 12).

The luciferase enzyme is encoded by luxA and luxB, whereas the enzymes responsible for the aldehyde biosynthesis are encoded by the three genes luxC, luxD and luxE. However, since aldehyde can rapidly diffuse across cellular membranes and is commercially available (e.g., Sigma), the genes encoding the synthesis of this substrate (luxCDE) are not an absolute necessity for bioluminescence and can be substituted by the addition of this compound exogenously. As such, in some embodiments, in order to generate a bioluminescent Gram-positive bacterium therefore, to provide a detectable signal it is necessary to ensure that the cell can synthesize a functional luciferase. In one embodiment, an expression construct comprising the lux operon is arranged as depicted in FIG. 1.

In another aspect of the invention, the invention includes an expression cassette comprising a polynucleotide encoding luxA, and luxB gene products, wherein (a) transcription of the polynucleotide results in a polycistronic RNA encoding both gene products, and (b) polynucleotide sequences comprising Gram-positive ribosome-binding site sequences are located adjacent the 5′ end of the luxA coding sequences and adjacent the 5′ end of the luxB coding sequences (e.g., SEQ ID NO: 12). In one embodiment, the expression cassette further comprises an insertion site 5′ to at least one of either the luxA or luxB coding sequences. The insertion site may, for example, further comprise a multiple-insertion site. In one embodiment, the multiple-insertion site is located 5′ to the luxA coding sequences. In a related embodiment, the multiple-insertion site is located 5′ to the luxB coding sequences. In another embodiment, the polynucleotide further encodes luxC, luxD and luxE gene products. The arrangement of the coding sequences for the lux gene products may be, for example, in the following relative order 5′-luxA-luxB-luxC-luxD-luxE-3′ or CDABE (FIG. 1), CDEAB, CABDE, or ABCDE.

In various embodiments, Gram-positive bacterial Shine-Dalgarno sequences are 5′ to all of the lux coding sequences. In one group of embodiments, transcription of the polynucleotide is mediated by a promoter contained in an expression enhancer sequence, such as Sa1-Sa6, e.g., Sa2 or Sa4. In another group of embodiments, transcription of the polynucleotide is mediated by a promoter contained in an enhancer sequence that can be Sp1, Sp5, Sp6, Sp9, Sp16 and Sp17, such as Sp16, or those disclosed in Table 1. In one embodiment, the coding sequences for luxA and luxB are obtained from Photorhabdus luminescens and comprise SEQ ID NO: 1 and SEQ ID NO: 3, respectively.

In yet another aspect, the invention includes an expression cassette comprising a polynucleotide encoding luxA, luxB, and luc gene products, wherein (a) transcription of the polynucleotide results in a polycistronic RNA encoding all three gene products, and (b) polynucleotide sequences comprising Gram-positive bacterial Shine-Dalgarno sequences are located adjacent the 5′ end of the lux coding sequences, and adjacent the 5′ end of the lux coding sequences. In one embodiment, the polynucleotide further encodes luxC, luxD and luxE gene products (e.g., FIG. 1). In another embodiment, Gram-positive bacterial Shine-Dalgarno sequences are located 5′ to all of the lux coding sequences or 5′ to luxA and/or luxC only. In yet other embodiments, transcription of the polynucleotide is mediated by a promoter contained in an enhances sequences, such as Sp1, Sp5, Sp6, Sp9, Sp16 and Sp17, e.g., Sp16, Sa1-Sa6, e.g., Sa2 or Sa4.

The expression cassette may further include a multiple-insertion site located adjacent the 5′ end of the lux coding sequences (FIG. 1). In various embodiments, the coding sequences for luxA and luxB are from Photorhabdus luminescens, and are optimized for expression in a low DNA G+C content host. In one embodiment, LuxA and LuxB are encoded by SEQ ID NO: 1 and SEQ ID NO: 3, respectively.

Eukaryotic luciferase (“luc”) is typically encoded by a single gene (de Wet, J. R., et al., Proc. Natl. Acad. Sci. U.S.A. 82:7870-7873, 1985; de Wet, J. R, et al., Mol. Cell. Biol. 7:725-737, 1987). An exemplary eukaryotic organism containing a luciferase system is the North American firefly Photinus pyralis. Firefly luciferase has been extensively studied, and is widely used in ATP assays. cDNAs encoding luciferases (lucOR) from Pyrophorus plagiophthalamus, another species of click beetle, have been cloned and expressed. (Wood, et al. Complementary DNA coding click beetle luciferases can elicit bioluminescence of different colors. Science 244:700-702, 1989.) This beetle is unusual in that different members of the species emit bioluminescence of different colors. Four classes of clones, having 95-99% homology with each other, have been isolated. They emit light at 546 nm (green), 560 nm (yellow-green), 578 nm (yellow) and 593 nm (orange).

Luciferases, as well as aequorin-like molecules, require a source of energy, such as ATP, NAD(P)H, a substrate to oxidize, such as luciferin (a long chain fatty aldehyde) or coelentrizine and oxygen. With the lux operon, the genes encoding the enzyme that synthesizes the aldehyde substrate are expressed contemporaneously with luciferase.

1.2.2. Lux Operons

In various aspects of the invention, different sources of lux genes can be used to provide sequence(s) which can be optimized for expression in low-GC organisms. In various embodiments, lux genes are optimized for A/T content to provide enhanced expression in low G/C Gram positive bacteria.

In other embodiments, a lux operon is modified to include mutation of the catalytic site of luxA to enhance the enzymatic activity of the luciferase at less partial pressure of oxygen. In other embodiments, the lux operon is mutated to shift the wavelength of the emitted light or to change the duration of the emission.

In various embodiments of the invention lux genes (e.g., lux ABCDE) are provided in an expression construct, and are provided via an episomal vector or integrated into the host genome. The order for the various lux genes can be CABDE, ABCDE, CDABE or CDEAB.

In one embodiment, the lux genes are provided in a construct as illustrated in FIG. 1, arranged in a CDABE fashion, where ribosome binding sites functional in desired bacteria are operatively linked to each gene (asterisks in FIG. 1). Furthermore, an inducible or constitutive promoter is provided.

In one embodiment, the lux polynucleotide cassette is optimized to match the codon usage of the bacterial species. In the case of Clostridium, the codon usage is optimized to 60-70% A/T content (or low G/C content). A gram-positive ribosome binding site (5′-AGGAGG-3′) is added 8-10 base pairs upstream of the start codon of each gene. Restriction enzyme sites are included for the rearrangement of genes. In one embodiment, transcription of the polynucleotide cassette is mediated by a promoter sequence. In another embodiment, a constitutive thiolase (thlA) promoter is included. Other embodiments will include inducible promoters specific for monitoring the production of compounds produced in fermentative, metabolic, or synthetic pathways e.g. the use bdhB to monitor butanol production in Clostridium.

Lux genes which can be utilized in the compositions and methods of the invention, are obtained from organisms including but not limited to Photobacterium phosphoreum, Vibrio salmonicida, Photobacterium leiognathi, Vibrio harvey, Photobacterium leiognathi, Vibrio fischeri, Photinus pyralis, Photorhabdus luminescens, formerly Xenorhabdus luninescens (Frackman, et al., Cloning, organization, and expression of the bioluminescence genes of Xenorhabdus luninescens. J. Bacteriol. 172″5767-5773, 1990; the sequence is available from GenBank under the accession number M90092.1). Furthermore, in contrast to luciferase from P. luminescens, other luciferases isolated from luminescent prokaryotic and eukaryotic organisms have optimal bioluminescence at lower temperatures. (Campbell, A. K. Chemiluminescence, Principles and Applications in Biology and Medicine. Ellis Horwood, Chichester, UK. 1988.)

A variety of other luciferase encoding genes have been identified including, but not limited to those disclosed in U.S. Pat. Nos. 5,670,356; 5,604,123; 5,618,722; 5,650,289; 5,641,641; 5,229,285; 5,292,658; 5,418,155; and de Wet, J. R., et al, Molec. Cell. Biol. 7:725-737, 1987; Tatsumi, H. N., et al, Biochim Biophys. Acta 1131:161-165, 1992; and Wood, K. V., et al, Science 244:700-702, 1989, all herein incorporated by reference. Such luciferase encoding genes may be modified by the methods described herein to produce polypeptide sequences and/or expression cassettes useful, for example, in Gram-positive microorganisms.

1.3. Transcription Regulatory Nucleotide Sequences/Promoters

In various embodiments of the invention, a transcription regulatory sequence is operably linked to gene(s) encoding a light emitting reporter(s) (e.g., in a expression construct). The transcription regulatory nucleotide sequences used in expression constructs of the invention are selected based on compatibility with the intended host. According to the present invention, the most preferred transcription regulatory nucleotide sequences are those from the host organism. For example, for the monitoring of the expression of acidogenic and solventogenic genes of C. acetobutylicum, the transcription regulatory nucleotide sequences include those from genes including but not limited to those listed in Table 1. In various embodiments, promoters are selected from genes including but not limited to butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase and butyryl-CoA dehydrogenase.

In various embodiments, constitutive or inducible promoters are selected for use in a host cell, for expression of an A/T optimized light emitting reporter (e.g., to monitor environmental pollutants). Depending on the host cell, there are hundreds of constitutive and inducible promoters which are known and that can be engineered with the optimized reporters of the invention. Examples of constitutive promoters include the int promoter of bacteriophage λ, the bla promoter of the β-lactamase gene sequence of pBR322, hydA or thlA in Clostridium, S. coelicolor hrdB, or whiE, the CAT promoter of the chloramphenicol acetyl transferase gene sequence of pPR325, Staphylococcal constitutive promoter P_{blaZ} and the like. Examples of inducible prokaryotic promoters include the major right and left promoters of bacteriophage (P_{L} and P_{R}), the trp, reca, lacZ, AraC and gal promoters of E. coli, the α-amylase (Ulmanen Ett at., J. Bacteriol. 162:176-182, 1985) and the sigma-28-specific promoters of B. subtilis (Gilman et al., Gene sequence 32:11-20 (1984)), the promoters of the bacteriophages of Bacillus (Gryczan, In: The Molecular Biology of the Bacilli, Academic Press, Inc., NY (1982)), Streptomyces promoters (Ward et at., Mol. Gen. Genet. 203:468-478, 1986), Staphylococcal cadmium-inducible P_{cad}-cadC promoters and the like. Exemplary prokaryotic promoters are reviewed by Glick (J. Ind. Microtiot. 1:277-282, 1987); Cenatiempo (Biochimie 68:505-516, 1986); and Gottesman (Ann. Rev. Genet. 18:415-442, 1984). Further examples of inducible promoters, such as in Clostridium species, include recA or recN gene promoters can be utilized which are part of the SOS repair system in Clostridium, or T5, CP25, P32, P59, P1P2 and PL promoters which can be linked to at least one operator selected from the group consisting of xylO, tetO, trpO, malO and λclO. See US Patent Application 2003-0027286.

In some embodiments, a promoter which is constitutively active under certain culture conditions, may be inactive in other conditions. For example, the promoter of the hydA gene from Clostridium acetobutylicum, expression is known to be regulated by the environmental pH. Therefore, in some embodiments, depending on the desired host cell, a pH-regulated promoter can be utilized with the expression constructs of the invention (e.g., FIG. 1 with hydA promoter driving expression of the optimized lux genes in response to variations in environmental pH). Other pH regulatable promoters are known, such as P170 functioning in lactic acid bacteria, as disclosed in US Patent Application No. 2002-0137140.

In general, to express the desired gene/nucleotide sequence efficiently, various promoters may be used; e.g., the original promoter of the gene, promoters of antibiotic resistance genes such as for instance kanamycin resistant gene of Tn5, ampicillin resistant gene of pBR322, and promoters of lambda phage and any promoters which may be functional in the host cell. For expression, other regulatory elements, such as for instance a Shine-Dalgarno (SD) sequence (e.g., AGGAGG and so on including natural and synthetic sequences operable in the host cell) and a transcriptional terminator (inverted repeat structure including any natural and synthetic sequence) which are operable in the host cell (into which the coding sequence will be introduced to provide a recombinant cell of this invention) may be used with the above described promoters.

Moreover, methods of identifying bacterial promoters can be practiced in selecting a promoter to be utilized in expression constructs of the present invention. Such methods are known, such as disclosed in US Patent Application No. 20060029958, U.S. Pat. No. 6,617,156. Through the analysis of the transcription regulatory nucleotide sequences, the appropriate primers can be designed so that the transcription regulatory nucleotide sequence of interest can be cloned from genomic DNA by use of the technique of polymerase chain reaction (PCR). The transcription regulatory sequences for genes from any desired host can be identified through the use of computational methods utilizing the sequenced genome of the host (e.g., genome of C. acetobutylicum ATCC 824 to obtain promoters therefrom). See, Paredes, C. J. et al. Transcriptional organization of the Clostridium acetobutylicum genome, Nuc. Acids Res. 32:1973-1981. Furthermore, sequences for many pathways are known and available through interne based services such as TIGR or the National Center for Biotechnology Information (NCBI, www.ncbi.nlm.nih.gov). The transcription regulatory nucleotide sequences can also be identified through standard molecular biology techniques such as cDNA primer extension using primers derived from the gene sequences of interest coupled with reverse transcription.

TABLE 1
Sources for Transcription Regulatory Nucleotide Sequences for Select Genes of C. acetobutylicum

| | | | IR | | |
|---|---|---|---|---|---|
| Gene ID | Direction | Annotation | Length | Description | Reference |
| CAC1742 | + | pta | 264 | Phosphotransacetylase | Boynton. Appl. Environ. |
| | | | | | Microbiol. 1996 |
| CAC1743 | + | askA | 11 | Acetate kinase | Boynton. Appl. Environ. |
| | | | | | Microbiol.1996 |
| CAC2708 | − | hbd | 104 | Beta-hydroxybutyryl-CoA | Boynton. J. Bacteriol. 1996 |
| | | | | dehydrogenase, NAD- | |
| | | | | dependent | |
| CAC2711 | − | bcd | 13 | Butyryl-CoA dehydrogenase | Boynton. J. Bacteriol. 199 |
| CAC2712 | − | crt | 175 | Crotonase (3-hydroxybutyryl- | Boynton. J. Bacteriol. 199 |
| | | | | COA dehydratase) | |
| CAC2873 | − | | 326 | Acetyl-CoA acetyltransferase | Stim-Herndon. Gene. 1995 |
| CAC3075 | − | buk | 27 | Butyrate kinase, BUK | Walter. Gene. 1993 |
| CAC3076 | − | ptb | 108 | Phosphate butyryltransferase | Walter. Gene. 1993 |
| CAC3298 | − | bdhB | 276 | NADH-dependent butanol | Walter. J. Bacteriol. 1992 |
| | | | | dehydrogenase B (BDH II) | |
| CAC3299 | − | bdhA | 147 | NADH-dependent butanol | Walter. J. Bacteriol. 1992 |
| | | | | dehydrogenase A (BDH I) | |
| CAP0035 | − | adhe | 476 | Aldehyde-alcohol | Fontaine. J. Bacteriol. |
| | | | | dehydrogenase ADHE1 | 2002 |
| CAP0078 | − | thil | 105 | Acetyl coenzyme A | Winzer. J. Mol. Microbiol. |
| | | | | acetyltransferase | Biotechnol., 2000 |
| | | | | (thiolase) | |
| CAP0162 | + | adhel | 666 | Aldehyde dehydrogenase | Nair. J. Bacteriol. 1994 |
| | | | | (NAD+) | Fischer. J. Bacteriol. |
| | | | | | 175:6959-6969, 1993 |
| CAP0163 | + | ctfa | 63 | Butyrate-acetoacetate COA- | Nair. J. Bacteriol. 1994 |
| | | | | transferase subunit A | Fischer. J. Bacteriol. |
| | | | | | 175:6959-6969, 1993 |
| CAP0164 | + | ctfb | 4 | Butyrate-acetoacetate COA- | Nair. J. Bacteriol. 1994 |
| | | | | transferase subunit B | Fischer. J. Bacteriol. |
| | | | | | 175:6959-6969, 1993 |
| CAP0165 | − | adc | 232 | Acetoacetate decarboxylase | Gerischer. J. Bacteriol. 172, |
| | | | | | 1990 |
| | | | | | Gerischer. J. Bacteriol. |
| | | | | | 174:426-433, 1992 |

a) Gene ID: Systematic gene code from TIGR;
b) Direction: Coding strand;
c) Annotation: Gene symbol according to TIGR;
d) IR length: Length of the upstream Intergenic Region;
e) Description: Description of gene function.

1.4 Fluorescent Proteins

In various embodiments of the invention, a recombinant cell comprises a high A/T sequence encoding a light emitting reporter as described herein (e.g., FIG. 1) and a second protein, such as fluorescent proteins described herein. As such, in some embodiments, two or more signal proteins can be used to shift the detectable wavelength of the emitted light from the recombinant host cell. For example, a low-GC host cell can comprise luciferase capable of expression in low-GC bacteria and GFP, where expression of both signals results in a different wavelength that is monitored. In various embodiments, the different signals can be on a bicistronic vector, under the control of the same or different promoters. Furthermore, vectors can be episomal or can provide constructs for integration into the host genome. Such bicistronic vectors are known in the art, such as disclosed in U.S. Patent Application NOs: 20060263882, 20060195935, 20060010506, 20050191723; U.S. Pat. Nos. 7,179,644; 7,090,976; 6,841,158; and 6,919,186.

Fluorescence is the luminescence of a substance from a single electronically excited state, which is of very short duration after removal of the source of radiation. The wavelength of the emitted fluorescence light is longer than that of the exciting illumination (Stokes' Law), because part of the exciting light is converted into heat by the fluorescent molecule.

Fluorescent molecules include small molecules, such as fluorescein, as well as fluorescent proteins, such as green fluorescent protein (GFP) (Chalfie, et al., Morin, et al.), lumazine, and yellow fluorescent proteins (YFP), (O'Kane, et al., Daubner, et al.) In nature, fluorescent proteins are often found associated with luciferase and function as the ultimate bioluminescence emitter in these organisms by accepting energy from enzyme-bound, excited-state oxyluciferin (Ward et al. (1979) J. Biol. Chem. 254:781-788; Ward et al. (1978) Photochem. Photobiol. 27:389-396; Ward et al. (1982) Biochemistry 21:4535-4540.) They can be used in the present system to increase the detector sensitivity to the bioluminescence generating system and to also shift the wavelength of the emitted light to a more appropriate wavelength for detection purposes.

The best characterized GFPs are those isolated from the jellyfish species Aequorea, particularly Aequorea victoria and Aequorea forskalea and the sea pansy Renilla reniformis. (Ward et al. Biochemistry 21:4535-4540; 1982; Prendergast et al. Biochemistry 17:3448-3453, 1978.) In A. victoria, GFP absorbs light generated by aequorin upon the addition of calcium and emits a green fluorescence with an emission wavelength of about 510 nm. (Ward et al. Photochem. Photobiol. Rev 4:1-57, 1979.)

Aequorea GFP encodes a chromophore intrinsically within its protein sequence, obviating the need for external substrates or cofactors and enabling the genetic encoding of strong fluorescence. (Ormo, M., et al. Crystal structure of the Aequorea victoria green fluorescent protein. Science 273:1392-1395, 1996.) The chromophore is centrally located within the barrel structure and is completely shielded from exposure to bulk solvent. Mutagenesis studies have generated GFP variants with new colors, improved fluorescence and other biochemical properties.

DNA encoding an isotype of A. victoria GFP has been isolated and its nucleotide sequence has been determined. (Prasher (1992) Gene 111:229-233.) Recombinantly expressed A. victoria GFPs retain their ability to fluoresce in vivo in a wide variety organisms, including bacteria (e.g., see Chalfie et al. (1994) Science 263:802-805; Miller et al. (1997) Gene 191:149-153), yeast and fungi (Fey et al. (1995) Gene 165:127-130; Straight et al. (1996) Curr. Biol. 6:1599-1608; Cormack et al. (1997) Microbiology 143:303-311).

Patents relating to A. victoria GFP and mutants thereof include the following: Chalfie, M., and Prasher, D. U.S. Pat. No. 5,491,084; Tsien, R., and Heim, R. U.S. Pat. No. 5,625,048; Tsien, R., and Heim, R. U.S. Pat. No. 5,777,079; Zolotukhin, S, et al. U.S. Pat. No. 5,874,304; Anderson, M., and Herzenberg, L. A. U.S. Pat. No. 5,968,738; Cormack, B. P., et al. U.S. Pat. No. 5,804,387; Tsien, R., and Heim, R. U.S. Pat. No. 6,066,476; Chalfie, M., and Prasher, D. U.S. Pat. No. 6,146,826; and Tsien, R., et al. U.S. Pat. No. 7,005,511.

Such relating to such fluorescent encoding genes may be modified by the methods described herein to produce polypeptide sequences and/or expression cassettes useful, for example, in Gram-positive microorganisms. In further embodiments, fluorescent proteins such as those described or disclosed above can be themselves modified for expression in desired host cells. For example, for expression in low-GC bacteria, nucleic acid sequences encoding such fluorescent proteins can be modified to for high A/T content (e.g., A/T optimization to about 62% to 75%).

1.5. Expression Cassettes

In one aspect of the invention, any of the nucleic acid constructs disclosed herein are comprised in an expression cassette. For example, a desired transcription regulatory nucleotide sequence for light-emitting reporter to be monitored is operably linked to a gene encoding a light emitting protein along with the appropriate translational regulatory elements (e.g., Gram-positive Shine-Dalgarno sequences), short, random nucleotide sequences, and selectable markers, to form what is termed an expression cassette. The methodologies utilized in making the individual components of an expression cassette and in assembling the components are well known in the art of molecular biology (see, for example, Ausubel, F. M., et al., or Sambrook, et al.) in view of the teachings of the specification. Examples of expression cassettes useful in the present invention include the gusA reporter cassette (Girbal, L., et al. supra) and the lacZ reporter cassette (Tummala, S. B. et al. Development and characterization of a gene expression reporter system for Clostridium acetobutylicum ATCC 824, Appl. Envir. Mircobiol. 65:3793-3799, 1999).

In one embodiment an expression cassette comprises a bacterial lux operon with the genes arranged in either the native orientation, luxCDABE (FIG. 1), or in a rearranged orientation, such as luxABCDE (U.S. Pat. No. 6,737,245). The bacterial lux operon is preferred over the eukaryotic luc operon because, the lux operon contains the genes for the endogenous production of an aldehyde substrate, unlike the luc operon. Therefore, the contemporaneous coproduction of luciferase and endogenous aldehyde substrate allows for real time measurement of bioluminescence by avoiding the need to add exogenous aldehyde before monitoring the bioluminescent signal strength as required with signal enzyme constructs utilizing the luc operon. One embodiment of the present invention uses a luciferase expression cassette wherein the lux operon from P. luminescens is operationally linked to the appropriate transcription regulatory nucleotide sequence for an enzyme in a fermentative pathway of C. acetobutylicum in a manner analogous to U.S. Pat. No. 6,737,245.

Another embodiment of this invention uses an expression cassette with a gene encoding a fluorescent protein operationally linked to the appropriate transcription regulatory nucleotide sequence for an enzyme in a fermentative pathway of C. acetobutylicum.

Any expression cassettes described herein optionally contain a site for insertion of known or unknown sequences. For example, an insertion site can typically be located 5′ to the luxB gene (i.e., between luxA and luxB).

1.5.1. Luciferase Expression Cassettes

In various embodiments, the present invention also includes expression cassettes that allow for expression of eukaryotic luciferase. In one embodiment, the luc expression cassette includes a polynucleotide encoding the luc gene product operably linked to a constitutively expressed promoter. In another embodiment, the luc expression cassette includes a polynucleotide encoding the luc gene product operably linked to an inducibly expressed promoter. In one embodiment, the promoter is obtained from a Gram-positive bacteria. In a further embodiment, the promoter is obtained from a low-GC Gram positive bacteria. In yet further embodiments, the promoter is obtained from an obligate or strict anaerobe Gram positive bacteria. In various such embodiments, an expression cassette can then be introduced into a suitable vector backbone, for example as a shuttle vector. In one embodiment, the shuttle vector includes a selectable marker and two origins of replication, one for replication in Gram-negative organisms, and the other for replication in Gram-positive organisms.

Appropriate promoters can be identified by any method known in the art in view of the teachings of the present specification. Furthermore, a variety of luciferase encoding genes have been identified including, but not limited to, the following: B. A. Sherf and K. V. Wood, U.S. Pat. No. 5,670,356, Kazami, J., et al., U.S. Pat. No. 5,604,123, S. Zenno, et al, U.S. Pat. No. 5,618,722; K. V. Wood, U.S. Pat. No. 5,650,289, K. V. Wood, U.S. Pat. No. 5,641,641, N. Kajiyama and E. Nakano, U.S. Pat. No. 5,229,285, M. J. Cormier and W. W. Lorenz, U.S. Pat. No. 5,292,658, M. J. Cormier and W. W. Lorenz, U.S. Pat. No. 5,418,155, de Wet, J. R., et al, (1987) Molec. Cell. Biol. 7:725-737; Tatsumi, H. N., et al, (1992) Biochim Biophys. Acta 1131:161-165 and Wood, K. V., et al, (1989) Science 244:700-702, all herein incorporated by reference.

1.6 Shuttle Vectors

Expression cassettes are then inserted into “shuttle vectors”, plasmids that can replicate in two or more hosts. A shuttle vector to be used with gram negative and gram positive organisms requires the shuttle vector to contain an origin of replication from each class. Examples of shuttle vectors include the pAUL-A vector (Chakraborty, et al. (1992) J. Bacteriol. 174:568 574), pMK4 and pSUM series (U.S. Pat. No. 6,737,245), and pIMP1 (Mermelstein, L. D., et al. Bio/Technology 10:190-195, 1992). Other vectors are well known to those skilled in the art and are readily available from catalogs.

1.7 Chromosomal Integration

Instead of transforming an organism with a plasmid, a signal enzyme can be integrated into a chromosome of the host. Use of chromosomal integration of the reporter construct offers several advantages over plasmid-based constructions, including greater stability, and the elimination of the use of antibiotics to maintain selective pressure on the organisms to retain the plasmids. In general, chromosomal integration is accomplished by the use of a DNA fragment containing the desired gene upstream from an antibiotic resistance gene such as the chloramphenicol gene and a fragment of homologous DNA from the target organism. This DNA fragment can be ligated to form circles without replicons and used for transformation. For example, the pfl gene can be targeted in the case of E. coli, and short, random Sau3A fragments can be ligated in Klebsiella to promote homologous recombination. In this way, ethanologenic genes have been integrated chromosomally in E. coli. (Ohta et al. Appl. Environ. Microbiol. 57: 893-900, 1991.)

The copy number of the integrated reporter can be controlled by the concentration of the antibiotic used in the selection process. For example, when a low concentration of antibiotics is used for selection, clones with single copy integrations are found, albeit at very low frequency. While this may be disadvantageous for many genes, a low copy number for luciferase may be ideal given the high sensitivity of the detectors employed in light measurement. Higher level expression can be achieved in a single step by selection on plates containing much higher concentrations of antibiotic.

1.8 Signal Enzymes that Parallel the Regulatory Control of the Monitored Enzymes

The expression of signal enzymes on shuttle vectors comprising a transcription regulatory nucleotide sequence for a native enzyme of a transformed host will naturally parallel that of the native enzyme that is to be monitored, since there will be two independent transcription regulatory nucleotide sequences present. Chromosomal integration will also result in parallel regulatory control, unless one is able to introduce the signal enzyme sequence in-line with the native gene.

1.9 Signal Enzymes having Regulatory Control In-line with the Monitored Enzymes

One way to place a signal enzyme under the same regulatory control as that of the native enzyme is to select the use of an operon located on an endogenous plasmid, like sol located on the pSOL1 megaplasmid. Here, the plasmid can be isolated, the operon excised and replaced by an expression cassette containing a new operon wherein the reporter gene is inserted in-line with the native gene to be monitored. Following transformation and amplification in an appropriate host, the plasmid can then be isolated and then used to transform a pSOL1 plasmid deficient strain of C. acetobutylicum.

### 2. Cell Culture

In one aspect of the invention a cell is engineered to contain a light-emitting reporter optimized for expression in the cell as described herein. Furthermore, as described herein, the light-emitting reporter allows real time monitoring of the cell to assess the physiological stage of the culture so that necessary modifications can be made to culture conditions (e.g., addition of nutrients, change of temperature/pH, etc). In this way, cultures can be monitored and optimized (e.g., to optimize growth conditions, optimize expression of a desired gene of interes, optimize production of a compound). Recombinant cells can be engineered using conventional techniques in the art, e.g., genome integration or plasmid transformation.

In various embodiments, where genes encoding a light emitting reporter are optimized to increase A/T content (e.g., from about 62% to 75%), the host cells selected are low-GC bacteria. In a further embodiment, low-GC bacteria are Gram positive bacteria. In yet another embodiment, the low-GC bacteria are strict or obligate anaerobe bacteria (e.g., C. acetobutylicum).

In various embodiments, a recombinant cell comprises a nucleic acid molecule comprising an expression control sequence operatively linked with a coding nucleotide sequence encoding a light-emitting reporter. Such recombinant cells are selected from a species including but not limited to Corynebacteria, Corynebacterium diphtheriae, Pneumococci, Diplococcus pneumoniae, Streptococci, Streptococcus pyogenes, Streptococcus salivarus, Staphylococci, Staphylococcus aureus, Staphylococcus albus, Myoviridae, Siphoviridae, Aerobic Spore-forming Bacilli, Bacillus anthracis, Bacillus subtilis, Bacillus megaterium, Bacillus cereus, Butyrivibrio fibrisolvens, Anaerobic Spore-forming Bacilli, Clostridium acetobutylicum (e.g., p262, ATCC43084), Clostridium acidisoli, Clostridium aciditolerans, Clostridium acidurici, Clostridium aerotolerans, Clostridium akagii, Clostridium aldenense, Clostridium algidicarnis, Clostridium algidixylanolyticum, Clostridium alkalicellulosi, Clostridium aminovalericum, Clostridium amygdalinum, Clostridium arcticum, Clostridium argentinense, Clostridium aurantibutyricum, Clostridium baratii, Clostridium botulinum, Clostridium bowmanii, Clostridium butyricum, Clostridium beijerinckii (e.g., ATCC 25752, ATCC51743), Clostridium cadaveris, Clostridium caminithermale, Clostridium carboxidivorans, Clostridium carnis, Clostridium celatum, Clostridium celerecrescens, Clostridium cellulolyticum, Clostridium cellulosi, Clostridium chartatabidum, Clostridium clostridioforme, Clostridium coccoides, Clostridium cochlearium, Clostridium cocleatum, Clostridium colinum, Clostridium difficile, Clostridium diolis, Clostridium disporicum, Clostridium drakei, Clostridium durum, Clostridium estertheticum, Clostridium fallax, Clostridium felsineum, Clostridium fervidum, Clostridium fimetarium, Clostridium formicaceticum, Clostridium ghonii, Clostridium glycolicum, Clostridium glycyrrhizinilyticum, Clostridium haemolyticum, Clostridium halophilum, Clostridium tetani, Clostridium perfringens, Clostridium phytofermentans, Clostridium piliforme, Clostridium polysaccharolyticum, Clostridium populeti, Clostridium propionicum, Clostridium proteoclasticum, Clostridium proteolyticum, Clostridium psychrophilum, Clostridium puniceum (ATCC43978), Clostridium puri, Clostridium putrefaciens, Clostridium putrificum, Clostridium quercicolum, Clostridium quinii, Clostridium ramosum, Clostridium roseum, Clostridium saccharobutylicum (e.g., ATCC BAA-117), Clostridium saccharolyticum, Clostridium saccharoperbutylacetonicum, Clostridium sardiniense, Clostridium stercorarium subsp. Thermolacticum, Clostridium sticklandii, Clostridium paradoxum, Clostridium paraperfringens, Clostridium paraputrificum, Clostridium pascui, Clostridium pasteurianum, Clostridium novyi, Clostridium septicum, Clostridium histolyticum, Clostridium hydroxybenzoicum, Clostridium hylemonae, Clostridium innocuum, Clostridium kluyveri, Clostridium lactatifermentans, Clostridium lacusfryxellense, Clostridium laramiense, Clostridium lentocellum, Clostridium lentoputrescens, Clostridium ljungdahlii, Clostridium methoxybenzovorans, Clostridium methylpentosum, Clostridium nitrophenolicum, Clostridium novyi, Clostridium oceanicum, Clostridium oroticum, Clostridium oxalicum, Clostridium tertium, Clostridium tetani, Clostridium tetanomorphum, Clostridium thermaceticum, Clostridium thermautotrophicum, Clostridium thermoalcaliphilum, Clostridium thermobutyricum, Clostridium thermocellum, Clostridium thermocopriae, Clostridium thermohydrosulfuricum, Clostridium thermolacticum, Clostridium thermopalmarium, Clostridium thermopapyrolyticum, Clostridium thermosaccharolyticum, Clostridium thermosulfurigenes, Clostridium tyrobutyricum, Clostridium uliginosum, Clostridium ultunense, Clostridium villosum, Clostridium viride, Clostridium xylanolyticum, Clostridium xylanovorans, Clostridium bifermentans, Clostridium sporogenes, Mycobacteria, Mycobacterium tubercolosis hominis, Mycobacterium bovis, Mycobacterium avium, Mycobacterium paratuberculosis, Actinomycetes (fungus-like bacteria), Actinomyces israelii, Actinomyces bovis, Actinomyces naeslundii, Nocardia asteroides, Nocardia brasiliensis, the Spirochetes, Treponema pallidium, Treponema pertenue, Treponema carateum, Borrelia recurrentis, Leptospira icterohemorrhagiae, Leptospira canicola, Spirillum minus, Streptobacillus moniliformis, Trypanosomas, Mycoplasmas, Mycoplasma pneumoniae, Listeria monocytogenes, Erysipelothrix rhusiopathiae, Streptobacillus monilformis, Donvania granulomatis, Bartonella bacilliformis, Rickettsiae (bacteria-like parasites), Rickettsia prowazekii, Rickettsia mooseri, Rickettsia rickettsiae, and Rickettsia conori.

The use of light-emitting reporters is applicable for the monitoring of all types of fermentative, metabolic, or synthetic pathways; expression of particular genes in a host cell; or the presence of a compound in the environment (e.g., mercury, metals, organic pollutants). The hosts may by “wild type” wherein they natively produce the desired target, or they may have already undergone mutagenesis and positive selection to overproduce the desired target. Alternatively, the host can be previously engineered to express enzymes required for the desired fermentative, metabolic, or synthetic pathway. This can be in the form of overexpressing the native enzymes required for the fermentative, metabolic, or synthetic pathways or the expression of heterologous enzymes required for a fermentative, metabolic, or synthetic pathway. Additionally, signal enzymes can be introduced simultaneously into the host cells with either native or heterologous fermentative, metabolic, or synthetic pathway enzymes. With simultaneous introduction, the signal enzymes can be on the same operon as the introduced fermentative, metabolic, or synthetic pathway enzymes or the signal enzymes can be located on different operons. Furthermore, the host can also be genetically modified so that expression of a necessary enzyme for a competing fermentative, metabolic, or synthetic pathway is down regulated or negated, thereby forcing substrate down the fermentative, metabolic, or synthetic pathway of interest.

With C. acetobutylicum, wild types strains contemplated for use with this invention include ATCC 43084 and ATCC 824 from the American Tissue Culture Collection (ATCC) and DSM 792 and DSM 1731 from the Deutsche Sammlung von Mikroorganismen and Zellkulturen GmbH, Germany. High butanol producing mutants of C. acetobutylicum contemplated for use with this invention include strains such as ATCC 55025, and ATCC 39058 from ATCC. Another high producing strain contemplated for use with this invent is B643. (Contag, P. R., et al, Cloning of a lactate dehydrogenase gene from Clostridium acetobutylicum B643 and expression in Escherichia coli. Appl. Environ. Microbiol. 56:3760-3765, 1990.) Enzymes anticipated to be overexpressed in C. acetobutylicum for the production of butanol include butyraldehyde dehydrogenase and butanol dehydrogenase. Enzymes of competing fermentative pathways anticipated to by down regulated or deleted in C. acetobutylicum include pyruvate decarboxylase, lactate dehydrogenase and acetate kinase.

The cell cultures of this invention are characterized in that they produce a target of a synthetic, metabolic, or fermentative pathway in commercially valuable quantities and they also produce a light emitting reporter that signals the status of target production. Conventional bioreactors and methods for culturing microorganisms to produce target products are known and are contemplated for use with the present invention methods and compositions.

2.1 Transformation of C. acetobutylicum

Numerous methods for the introduction of nucleic acid constructs of the invention into cells or protoplasts of cells are known to those of skill in the art and include, but are not limited to, the following: conjugation, viral vector-mediated transfer and electroporation.

Electroporation is the preferred method of transforming C. acetobutylicum. Ideally, electrocompetent C. acetobutylicum cells prepared from mid-logarithmic growth phase are used. Following electroporation, cells are incubated at 37° C. in an appropriate broth, like 2×YT broth while under a nitrogen atmosphere. Following a recovery period, the cells are transferred to an anaerobic glovebox, and serial dilutions are then plated on nutrient plates like 2×YT agar plates that are supplemented with the requisite antibiotic concentration.

2.2 Detection of Clones with Luciferase Containing Light Emitting Reporter Constructs

Colonies of microorganisms that containing nucleic acid constructs derived from the complete luxCDABE operon, can be identified by manual visual inspection in a darkened room or by the use of an image detection system such as one that incorporates a charge coupled device (CCD) camera, screening clones with a luminometer or through standard molecular biology techniques. Since oxygen is required for the bioluminescence reaction, plates may need to be exposed to low concentrations of oxygen in order to detect positive colonies. The expression cassettes derived from luc and luxAB require the addition of an exogenous substrate in order to produce light. In one embodiment of the present invention, the substrate is an aldehyde such as decanal. When administered to cells, aldehyde may be applied in the atmosphere surrounding the culture media as a vapor or directly to the culture media.

In another embodiment, the selectable marker may comprise nucleic acid sequences encoding for a reporter protein, such as, for example, green fluorescent protein (GFP), DS-Red (red fluorescent protein), acetohydroxyacid synthase (AHAS), beta glucoronidase (GUS), secreted alkaline phosphatase (SEAP), beta-galactosidase, chloramphenicol acetyltransferase (CAT), horseradish peroxidase (HRP), luciferase, nopaline synthase (NOS), octopine synthase (OCS), or derivatives thereof, or any number of other reporter proteins known to one skilled in the art.

In certain embodiments, commercially valuable quantities of a target include those targets produced in 100 l fermentors. In other embodiments, commercially valuable quantities of a target are produced in fermentors with 100 to 500 l capacity. In still further embodiments, commercially valuable quantities of a target are produced in fermentors of 500 l to 1,000 l capacity. In still other embodiments, commercially valuable quantities of a target are produced in fermentors of 1,000 l to 2000 l capacity. In certain other embodiments, commercially valuable quantities of a target are produced in fermentors with 2,000 l to 5,000 l capacity. In other embodiments, commercially valuable quantities of a target are produced in fermentors with 5000 l to 10,000 l capacity. In still other embodiments, commercially valuable quantities of targets are produced in fermentors with 10,000 l to 50,000 l capacity. In certain other embodiments, commercially valuable quantities of targets are produced in fermentors with 50,000 l to 200,000 l capacity. In still further embodiments, commercially valuable quantities of targets are produced in fermentors with 200,000 l to 400,000 l capacity. In certain embodiments, commercially valuable quantities of targets are produced in fermentors with 400,000 l to 800,000 l capacity. In still other embodiments, commercially valuable quantities of targets are produced in fermentors with 800,000 l to 1,600,000 l capacity. In certain embodiments, commercially valuable quantities of targets are produced in fermentors with 1,600,000 l to 3,200,000 l capacity.

### 3. Methods of Monitoring and Regulating

During growth and culture of microorganisms, the velocity of various biochemical pathways change, shifting the rate of production of various targets. For example, in the batch culture of C. acetobutylicum, the initial production of acids, such as acetate and butyrate, decreases the pH of the culture, however, once the concentration of undissociated butyrate reaches 9 mM, a shift occurs wherein the C. acetobutylicum reassimilates the secreted acids and switches to the production of solvents such as butanol and acetone. Butanol has a toxic effect upon the cells and its accumulation eventually inhibits the expression of the enzymes that produce it. By placing reporters at strategic points in various biochemical pathways one can monitor the status of these pathways and, if desired, one can “poise” the culture conditions to induce and maintain a state that produces the maximum amount of a product. In the case of an observed inhibitory effect of butanol on the culture, the removal of butanol from the fermentation broth can commence or water or culture media can be added to the fermentor to dilute the accumulated butanol below the inhibitory threshold.

The status of a biochemical pathway is signaled by the intensity of the signal being produced by the reporter. This, in turn, reflects the transcriptional activity of signal enzyme construct and the pathway enzyme that relies on the same promoter sequence as the signal enzyme construct. Light emitting reporters are particularly attractive because they produce a signal in real time that correlates with the degree of gene expression providing immediate information regarding the status of a fermentative, metabolic, or synthetic pathway. The use of signal enzymes in the culture of microorganisms such as C. acetobutylicum allows culture conditions to be adjusted immediately to reverse a decline, maintain or induce high productivity. The use of a light emitting report can provide information regarding culture conditions before the fermentative, metabolic or synthetic compounds of interest are detectable in the culture by other means such as HPLC analysis of culture media or mass spectrometry analysis of culture offgas.

The ability to gather immediate information on the expression of a pathway gene of interest also allows for the rapid and efficient development of culture media for the production of a fermentative, metabolic, or synthetic compound of interest. Instead of waiting for a compound to be produced and secreted into the culture media in sufficient quantities that will allow for it's detection and quantification, productivity information can be obtained in real-time by the analysis of the expression of a light emitting reporter. Productivity changes caused by changes to the culture media conditions can be immediately identified, saving time and reducing uncertainty. Similar productivity information can be obtained for physical changes to the culture conditions such as temperature, pH, oxygen partial pressure, or dilution rates.

Culture conditions can be identified by measuring the light produced by a light emitting reporter, changing one or more culture parameters and then measuring the change in light production. Depending on the pathways being monitored and the fermentative, metabolic, or synthetic compound of interest, an increase in signal strength may indicate that more compound of interest is being produced when the signal enzyme construct utilizes an inducible promoter required by an enzyme in that pathway. Alternatively, if the signal strength increases, this can indicate that productivity is decreasing if the light emitting reporter utilizes an inducible promoter required by an enzyme in a competing pathway. A decrease in the signal strength of a light emitting reporter can similarly indicate a decrease or increase in productivity depending on whether the inducible promoter used by the signal construct is also used by an enzyme in the pathway of interest or by an enzyme in a competing pathway.

An alternative way to develop or test culture conditions is to run multiple fermentations, each having one or more change in media composition, feedstock, feed rate, physical parameters and the like compared to a control fermentation. The light for each culture can be monitored and compared with the control run and with each other. In this way, conditions that increase expression of a signal construct that utilizes an inducible promoter in a pathway of interest are identified for further testing to confirm the predicted increase in productivity. In this way large numbers of culture conditions can be rapidly screened. This methodology can be adapted for high throughput screening including the use of multiple well culture plates such as 96 well plates.

A further way to develop or test culture conditions is a hybride between the two methods detailed above. Multiple small cultures can be monitored for light emission and then the culture conditions can be changed to see what influence they have on culture productivity. For example, if during the screening process a culture that has a 1% higher concentration of glucose than the control is identified as being more productive, additionally glucose can be added step-wise, incrementally, or at an exponentially increasing rate to see how high of expression of the light emitting reporter can be achieved. Using this method will result in the rapid identification of productive culture conditions.

A further use of the light emitting reporter is for the screening of mutants. A production strain with a light emitting reporter can be mutagenized and individual colonies isolated. These isolated mutants can then be grown in culture and their expression of the light emitting reporter measured. Mutants can be rapidly screened through the use of multiple well culture plates like 96 well plates and a bioluminescence plate reader. Cultures that have higher expression than the parent strain indicate mutants that potentially have higher productivity than the parent strain. Multiple rounds of mutagenesis and screen can be quickly performed to generate high production strains.

3.1. Detection of Light in a Culture

This invention contemplates several ways in which to measure light in a microbial culture. Conventionally fermentors can have a port hole positioned on the side of the tank so that the port hole will be beneath the initial level of the fermentation broth. A means of detecting light such as a photomultiplier tube (PMT), or a CCD camera can then be mounted outside of the port hole, but positioned to detect any light that is emitted through the port hole window. Alternatively, a detector, or a light guide can be placed inside of the fermentor through the port hole prior to sterilization of the fermentor.

Additionally, a stream of the culture media can be continuously drawn off the fermentor and directed to a light detection apparatus. There the sample stream can be either intermittently or continuously passed through a flow cell positioned inside the light detection apparatus. Here, a mixing chamber can be place so that ATP or oxygen can be added to the sample stream if it is needed to enhance the luminescence of the media. Alternately, a diluent can be added to the sample in the mixing chamber to decrease the signal intensity if needed.

Furthermore, samples can be drawn off the fermentor periodically, through a sampling port either manually or automatically, and then analyzed for luminescence.

3.2. Processing of the Light Signal

An important aspect of the present invention is the use of a highly sensitive means to enable the rapid measurement of bioluminescence from fermentation broth so that the obtained signal can be used for real time monitoring and control of the culture. The device needs to be able to detect and count individual photons and accumulate the total count over time like in the manner of a scintillation counter. The most sensitive counting device employs a photomultiplying tube (PMT) wherein light entering the PMT excites electrons in the photocathode resulting in the emission of photoelectrons that as they are accelerated towards the detector unleash a growing cascade of electrons that are detected. Numerous PMTs are available from suppliers such as Hamamatsu.

Less sensitive devices include charge coupled device (CCD) cameras. These can be cooled to reduce background noise or they can contain microchannel intensifiers that function in a manner analogous to a PMT to boast the signal generated by incident photons. An exemplary microchannel intensifier-based single-photon detection device is the C2400 series, available from Hamamatsu.

Both PMTs and CCDs are available in modules for convenience that contain all the need power sources and electronic circuitry. For example a PMT module usually contains a high voltage power supply, voltage divider circuitry, signal conversion circuitry, photon counting circuitry, CPU interface and a cooling device integrated into a single package. Software is readily available that allows integration of the photon count signal with a computer thereby allowing the signal to be used in an algorithm for the monitoring and control a fermentation process.

3.3. Determining Status of the Biochemical Pathway: Computer Software

Determining the status of a biochemical pathway depends on the nature of signal enzyme on which the reporter reports. The signal can be positively or negatively correlated with the production of the target depending on whether the signal enzyme catalyzes a transformation toward the target or toward a branch leading either to another end product or to an intermediate that is recycled back to the pathway. Between these two alternatives, the absolute level of the signal provides information about the production of the desired product, and the kinetics of the signal, that is the change in intensity over time, also provides information about whether product production is increasing or decreasing. Therefore, both the absolute level of signal strength and the kinetics of signal strength can be usefully measured and used in this invention.

While this information can be processed and acted upon by a person, in certain embodiments the information is processed by a computer. Thus, software of this invention will include code that receives as input data concerning the level of signal from each of the reporters, code that executes an algorithm that determines the state of the culture as a function of (at least) this level or level, and code that determines how the culture conditions should be changed to poise that culture at a desired state, and code that instructs the system to made the appropriate changes to the culture to achieve this condition, be it adjusting temperature, adding nutrients, removing a product from culture, decreasing the density of the culture, or any other change that will shift the culture to a desired state.

3.4. Regulating Pathway Activity in Culture

The ability to monitor enzyme expression and hence, activity along fermentative, metabolic, or synthetic pathways, in real-time by the use of signal enzymes provides the operator or fermentation process controller with the ability to adjust conditions to “poise” the culture in a particular phase for maximum productivity of the desired product. One way to utilize the real time signaling capability of signal enzymes to control a culture is to adopt the real time signal methodologies used to control common high cell density E. coli fermentations. Here, cells are typically grown in batch mode to an intermediate cell density following which feeding strategies are initiated. The feeding strategies can be classified into two major categories: open-loop (non-feedback) and closed-loop (feedback). (U.S. Pat. No. 6,955,892.) The open-loop feeding strategies are typically pre-determined feed profiles for carbon/nutrient addition. Commonly used feed schedules include constant or increasing feed rates (constant, stepwise or exponential) in order to keep up with the increasing cell densities. While these simple pre-determined feed profiles have been applied successfully in certain cases, the major drawback is the lack of feed rate adjustment based on metabolic feedback from the culture. Therefore, the open-loop feeding strategies can fail by overfeeding or underfeeding the culture when it deviates from its “expected” growth pattern.

The closed-loop feeding strategies, on the other hand, typically rely on measurements that indicate the metabolic state of the culture. The two most commonly measured online variables for E. coli are dissolved oxygen (DO) concentration and pH. With DO monitoring, a rising DO signifies a reduction of oxygen consumption that in turn is based on nutrient limitation or depletion. When the DO rises above a threshold value or the rate of change is above a threshold value, the process controller will increase the nutrient feed rate. Conversely, when the DO drops below the desired set point or the rate of change is above a threshold value, the process control will reduce the nutrient feed rate to reflect metabolic demand. Similarly, changes in culture pH or the rate of change of a culture pH can be used alone or in combination with DO measurements to adjust the rate at which nutrient feed is added to the fermentor.

Since signal enzymes provide real time status of the metabolic activity of the culture, the same process control algorithms used with DO and pH control of conventional high density cell culture systems can be adopted for use with signal enzymes systems. This would be particularly advantageous in the monitoring of anaerobic cultures where DO monitoring is impossible. Taking butanol production in C. acetobutylicum as an example, once the culture is firmly into the solventogenic phase, the majority of intermediates for butanol production will come from the continued metabolism of feedstock like glucose. Use of a signal enzyme towards the end of the butylic pathway such as bdhB, an aldehyde-alcohol dehydrogenase that reduces butyraldehyde to butanol, provides status as to the production of butanol and hence, the metabolic rate of the culture. The signal strength and rate of change of the signal strength can then be used to control the feed rate of the culture in much the way as it is done by DO monitoring in E. coli cultures. This can be done in C. acetobutylicum batch culture by monitoring the initial expression of the signal enzyme as the culture starts to produce solvents. There may be an initial increase in the signal strength as organic acids from the acidogenic phase are reassimilated and these intermediates are shunted down the butylic pathway. As the concentration of these acids decrease in the fermentation media, the transcription rate of the butylic pathway enzymes may decrease in parallel signaling the process controller to initiate feeding of the culture or to increase the existing feed rate. Thereafter, an increasing signal strength indicates that butanol production is increasing and therefore, so is the metabolic rate of the culture. The process control would then increase the feed rate incrementally while continuing to monitor the signal strength of the enzyme. If the signal strength continues to increase, the process controller can continue to increase the feed rate so long as the rate of change of the signal strength of the signal enzyme is increasing. If a decrease in the rate of change for the signal strength of the signal enzyme is noted, the process controller will reduce the feed rate in order not to over feed the culture and cause substrate inhibition and a reduction in butanol production rate. By continued monitoring of the signal enzyme signal and adjusting of the feed rate to reflect the information provided by the signal enzyme, the culture will be place in a state of maximum butanol productivity.

Alternatives to batch culture are fed-batch and continuous culture. With continuous culturetypically, fermentation broth is simultaneously removed from the fermentor and fresh nutrients or water is added to maintain fermentor volume and desired cell density. Since a continuous fermentation process represents a relatively steady state it can also be monitored and controlled through the use of one or more signal enzymes. Any decrease or increase in signal strength represents a deviation away from the preexisting steady state and depending upon the desired fermentation parameters, such signaling may indicate to the operator or process controller that it is time to adjust the fermentation conditions. The use of a light emitting reporter allows for the monitoring of real time changes in culture conditions and avoids the need to wait for product to accumulate in the media or offgas in concentrations or in changes in concentrations that are detectable. The requirement for the continuous removal of fermentation broth in maintaining a steady state provides a ready means to employ in-line measurements of signal enzymes monitoring.

Signal enzymes can also be used for monitoring catabolite repression in a fermentative, metabolic, or synthetic pathway. Some enzymes are sensitive to the concentration of catabolite present, wherein the catabolite is able to bind to the operon for the enzyme and block the transcription of the gene. As catabolite concentration increases the rate of gene transcription for the enzyme decreases. With the use of a signal enzyme construct that utilizes the same transcription regulatory nucleotide sequence, signal strength of the signal enzyme will decline proportionally. When the fermentation process controller detects a drop in the signal strength of the signal enzyme, the process control can take action to counter the accumulation of the repressive catabolite. For example, if the catabolite is a target that is secreted into the media, the process controller can initiate the removal of the target from the culture media. If the catabolite is an intermediary, the intracellular concentration of the repressor can be reduced by increasing the total volume of the culture through the addition of water or fresh culture media.

The use of multiply signal enzyme constructs each with a different inducible promoter allows the simultaneous monitoring of one or more fermentative, metabolic, or synthetic pathways. If two or more pathways are present in an organism, then by placing one signal enzyme construct in each pathway one can then determine which pathways are active and also indicate the strength of the activity, thereby providing the opportunity to adjust the culture conditions to selectively increase or decrease the flux of intermediates down a particular pathway. For example, with C. acetobutylicum, there are two growth phases in batch culture, first an acidogenic phase in which organic acids accumulate in the culture media, followed by the solventogenic phase in which the organic acids are reassimulated and then shunted down the butylic, acetogenic, and ethanolic pathways along with metabolic intermediates produced by the breakdown of feedstock like glucose. In the acidogenic phase of a batch culture, if a signal enzyme along the solventogenic pathway starts indicating activity along that pathway, the operator or process controller can if desire, add pyruvate to the culture media as a substrate. This induces the expression of acidogenic enzymes thereby prolonging the acidogenic phase. (Junelles A. M. et al. Effect of pyruvate on glucose metabolism in Clostridium acetobutylicum. Biochimie. 69:1183-1190, 1987.) This could be done to provide more organic acids for later reassimulation and conversion to solvents thereby increasing solvent yields.

Similarly, if temperature or pH is found to influence the productivity of a particular fermentative, metabolic, or synthetic pathway, then the use of a signal enzyme could be used to maximize productivity. For example, if a particular strain of C. acetobutylicum, is found to produce more organic acids at one temperature, but a greater concentration of butanol relative to the other solvents at another temperature, then the use of a signal enzyme could indicate when the solventogenic shift has occurred so that the temperature of the culture can be adjusted in a timely manner for maximum butanol productivity.

The general metabolic health of a culture can be determined through the use of a light emitting reporter with a constitutive promoter. Changes in the observed light will reflect both changes in cell mass and metabolic flux in the individual organisms. As a culture enters the exponential growth phase, the amount of emitted light will correspondingly increase. Once the growth rate platues and metabolic activity slows, the measured signal strength will decrease. Since the sensitivity of light detecting instrumentation is very high, the use of light emitting reporters can provide information on a culture's growth rate much earlier than spectometry (OD), thereby providing growth information while the culture is in the lag phase.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Making Light-Emitting Expression Vectors

### Bacterial Strains, Media, and Growth Conditions

Clostridium beijerinckii ATCC 51743, C. acetobutylicum 824 and Escherichia coli DH5a pJIR418 were obtained from the American Type Culture Collection. C. beijerinckii and C. acetobutylicum were grown anaerobically at 35° C. in either yeast extract medium (YEM) from spore stocks, P2 medium for analysis of fermentation at 5 ml and 15 L scale, or 2×YTG for preparation of electrocompetent cells. Media was supplemented with 50 μg/ml of erythromycin when necessary to select for a plasmid. Reinforced clostridial medium (RCM) was used for growth on agar plates. Construction of the optimized lux operon for expression in Clostridium

The lux operon encoding luxCDABE was optimized for low G+C organisms using the lux sequence from Photorhabdus luminescence SEQ ID NO. 11 (GenBank #M90092.1). The lux operon was constructed by by Codon Devices (Cambridge, Mass.) and cloned into the pUC19 vector and designated pUC19-luxCDABE or lux*, FIG. 1. The AT richness of the codon was increased to 69% without changing the amino acid sequence. The gram positive ribosome binding site (Shine-Dalgarno sequence), 5′-AGGAGG-3′, was included 8-10 base pairs upstream of the ATG start site of each of the five genes. Restriction enzyme sites were designed flanking each gene and the operon for cloning. The optimized lux operon was then cloned into the pJIR418 vector to create pJIR418-lux* (lacking a Clostridium promoter). Two further constructs were created, one to measure the constitutive expression of light, while the other was under the control of an inducible promoter. For constitutive expression of the lux genes, the thiolase (thl) promoter was amplified from C. acetobutylicum 824 genomic DNA (ATCC) with the following primers: thl Forward 5′-CATTAGGATCCTAGAATGAAGTTTCTTATGCAC-3′ and thl Reverse 5-CATTAGCTCGAGAAATTTTGATACGGGGTAAC 3′. Restriction enzyme sites BamHI (forward primer) and XhoI (reverse primer), shown underlined, were included for cloning. The thl promoter was then cloned into pUC19-luxCDABE to create pUC19-thl-luxCDABE or thl-lux*. This operon was then cloned into the pJIR418 plasmid to create pJIR418-thl-lux*.

For investigating regulated expression, the terminal enzyme in the formation of butanol by C. acetobuylicum, butanol dehydrgenonase B (bdhB), was used. The bdhB promoter was amplified from C. acetobutylicum 824 genomic DNA with the following primers: bdhB Forward 5′-CATTAGGATCCTAAATGCAGAGGATGTTCTTGAG-3′ and bdhB Reverse 5′-CACTTTAACCCCTCGAGTTTAG-3′. Restriction enzyme sites BamHI (forward primer) and XhoI (reverse primer), shown underlined, were included for cloning. The bdhB promoter was then cloned into pUC19-luxCDABE to create pUC19-bdhB-luxCDABE or pUC19-bdhB-lux*. This operon was then cloned into the pJIR418 plasmid to create pJIR418-bdhB-lux*.

Thus three constructs were created: pJIR418-lux* (control), pJIR418-thl-lux* (constitutive), and pJIR418-bdhB-lux* (inducible).

Electroporation of E. coli

Standard techniques known in the art where used to transform E. coli with plasmid constructs.

Electroporation of C. beijerinckii and C. acetobutylicum

The pJIR418 plasmid constructs were electroporated into C. beijerinckii or C. acetobutylicum following the method of Oultram, et al (Oulteram et al., Introduction of plasmids into whole cells of Clostridium acetobutylicum by electroporation, FEMS Microbiology Letters 56, 83-88, 1988). Briefly, small cultures were started from a spore stock and grown to mid log phase in YEM. A 100 ml culture was inoculated 1:10 from the mid log phase culture in 2×YTG and grown to an OD of 0.8. Bacteria were pelleted, suspended in electroporation buffer, and electroporated with 1 μg of the plasmid construct. After electroporation, bacteria were suspended in 2×YTG and incubated for 4 h. Samples were plated on RCM plates supplemented with 10 μg/ml erythromycin to select for the plasmid.

### Fermentation Experiments

Batch or continuous fermentations were performed at the 15 L scale. A 1:20 inoculum was use to inoculate the P2 media supplemented with 4% glucose. Nitrogen was sparged to obtain anaerobic conditions. pH, redox, and temperature were measured. CO₂, H₂, O₂, and butanol production were measured by mass spectrometry. Butanol production was additionally measured by HPLC.

### Bioluminescence Imaging

Bacterial fermentations, cultures and plates were analyzed for bioluminescence using an In Vivo Imaging System (IVIS) (Caliper Life Science, Hopkinton, Mass.). Samples from the anaerobic incubator were exposed to oxygen prior to imaging. Samples from liquid cultures were imaged in triplicate in 100 μl volumes in microtiter plates for 2-5 minute integration times. The bioluminescence image is overlayed on the black and white photograph of the sample. Total flux (p/s) is determined for each well by creating a region of interest.

Testing of the Optimized Lux Cassette in E. coli

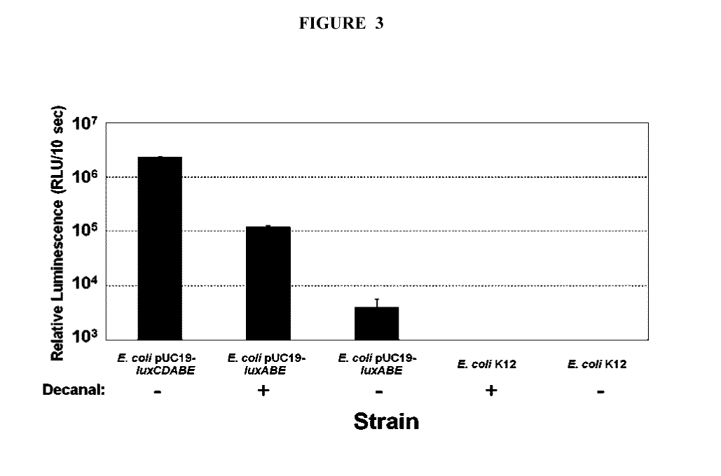

The functioning of the high A/T optimized lux* cassettes were first demonstrated in E. coli K12. FIG. 3. Cells were transformed with either pUC19-luxABE or pUC19-luxCDABE. Nontransformed E. coli K12 served as a control. Relative luminescence was measured using a luminescence plate reader. The complete lux operon (pUC19-luxCDABE) produced 10⁶ RLU/10 sec without the need for the addition of substrate. The optimized partial lux operon (pUC19-luxABE) produced luminescence at 10⁵ RLU/10 sec with substrate and 10³ RLU/10 sec without substrate. Non-transformed E. coli K12 only demonstrated nominal background lumenscence.

### Testing of the Optimized lux Cassettes in Clostridium Species

After demonstrating that the optimized lux cassettes retained function when expressed in E. coli, a constitutive promoter, the thiolase (thl) promoter and an inducible promoter butanol dehydrgenonase B (bdhB) the terminal enzyme in the butanol pathway were amplified from C. acetobutylicum 824 genomic DNA by PCR and then cloned into the pUC19 plasmid upstream from the lux sequence to create two additional plasmids. Subsequently, these three cassettes were cloned into the pJIR418 vector creating pJIR418-lux* (control), pJIR418-thl:-ux* (constitutive), and pJIR418-bdhB-lux* (inducible).

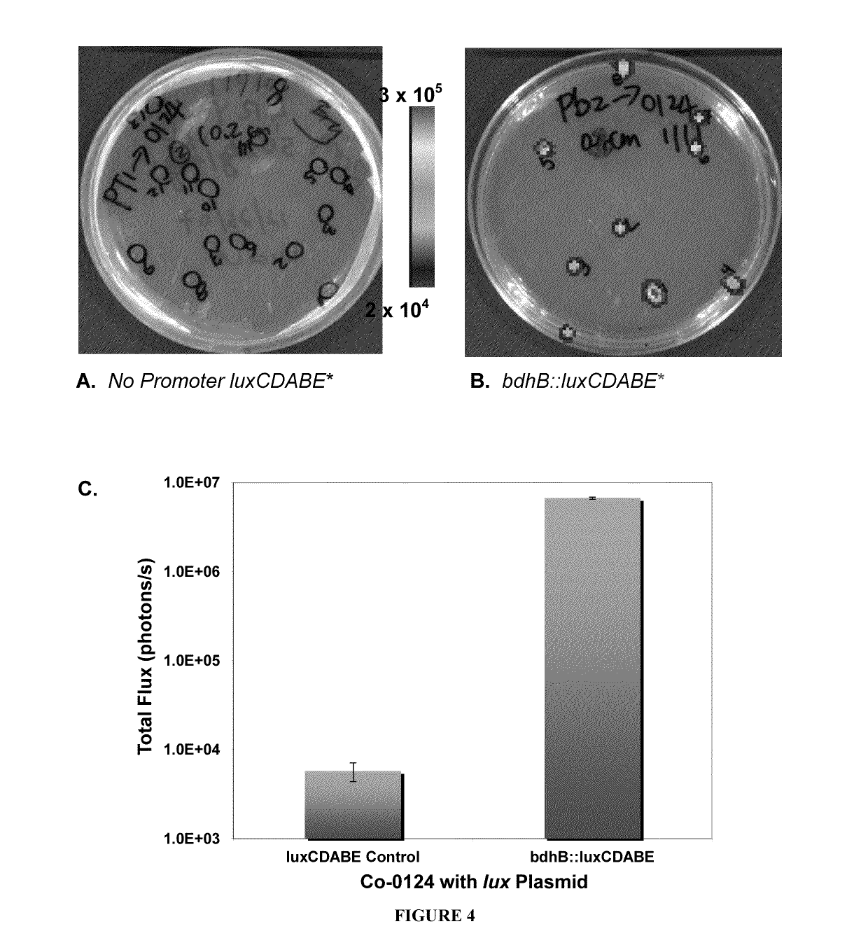

The functionality of an optimized pJIR418-lux* cassette was then demonstrated in a low G+C organism, C. beijerinckii. FIG. 4. Here, colonies of electroplated cells that expressed the optimized lux operon containing the inducible promoter could be discerned with IVIS while transformants that expressed the optimized lux operon without a promoter had a lumescence comparable to the background level. In liquid culture, the transformants with the inducible promoter demonstrated a 3-log difference in luminescence compared to the cells that expressed the lux operon without a promoter.

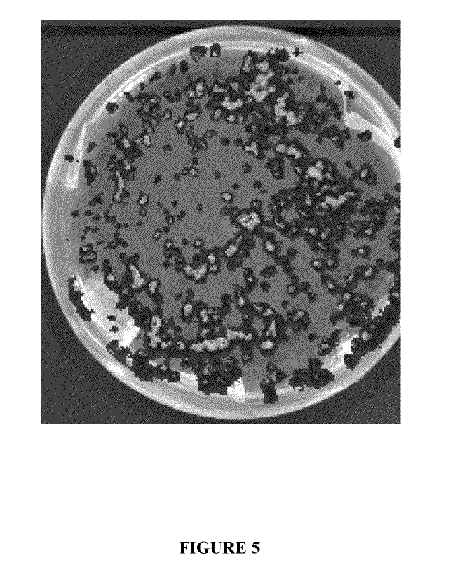

Functionality of the lux operon with the inducible promoter was then demonstrated in another low G+C organism, C. acetobutylicum. FIG. 5.

### Demonstration of Optimized lux Cassettes in Batch Culture

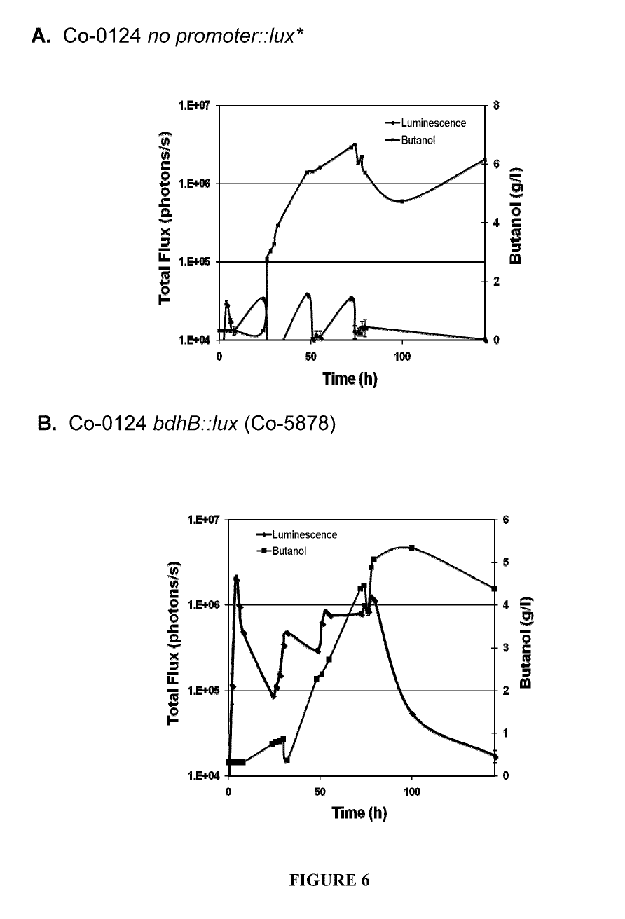

Next the correlation of bioluminescence with butanol production was measured in small batch cultures of C. beijerinckii over time. FIG. 6. Samples were taken periodically and the bioluminescence total flux (photons/sec) was detected with IVIS while butanol formation was determined by HPLC. (A) The Clostridium strain Co-0124 with the promoterless lux construct produced appreciable levels of butanol but light production was nominally above background levels (˜10⁴ p/s). (B) Strain Co-5878 (Co-0124 transformed with the inducible construct, pJIR418-bdhB-lux*) demonstrated a 100-fold increase in light production that preceded any measurable increase in butanol formation. Once butanol formation ceased there was a dramatic decrease in light production.

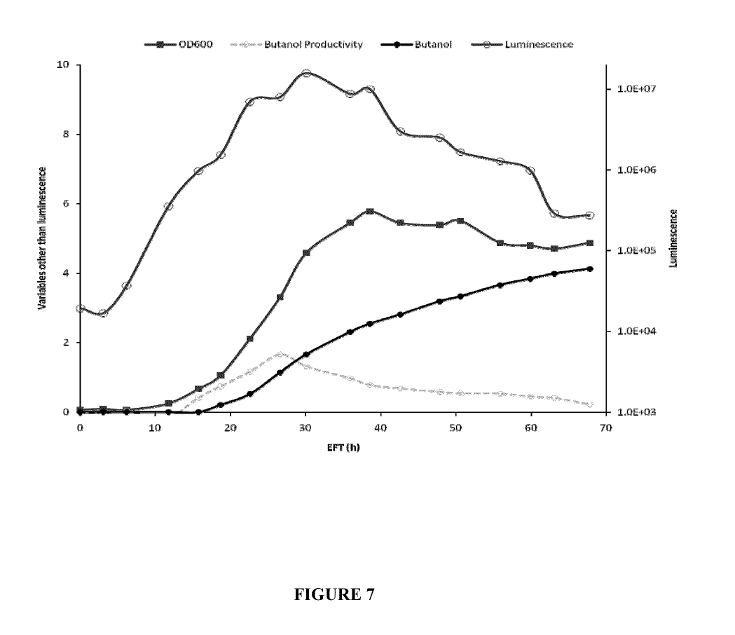

With transformants having the constitutive promoter thiolase (thl, pJIR418-thl::lux*) bioluminescence correlated with the growth rate of the culture rather than with butanol productivity. FIG. 7. The detection of bioluminescence preceded that of butanol, but mirrors the increase in OD demonstrating a rapid increase during the exponential phase of growth. Bioluminescence peaks prior to the peak in OD and then gradual declines during the platue phase of cell growth. Butanol, on the other hand, continues to accumulate.

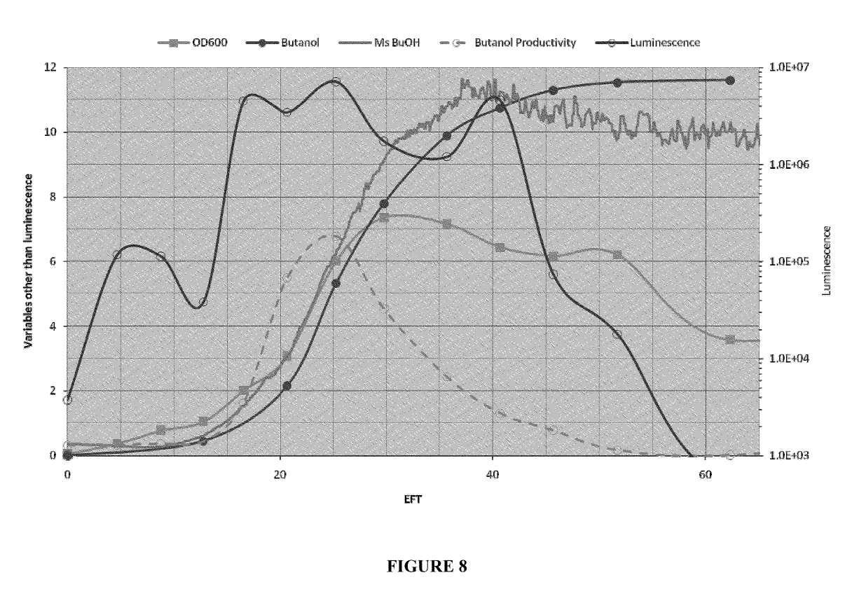

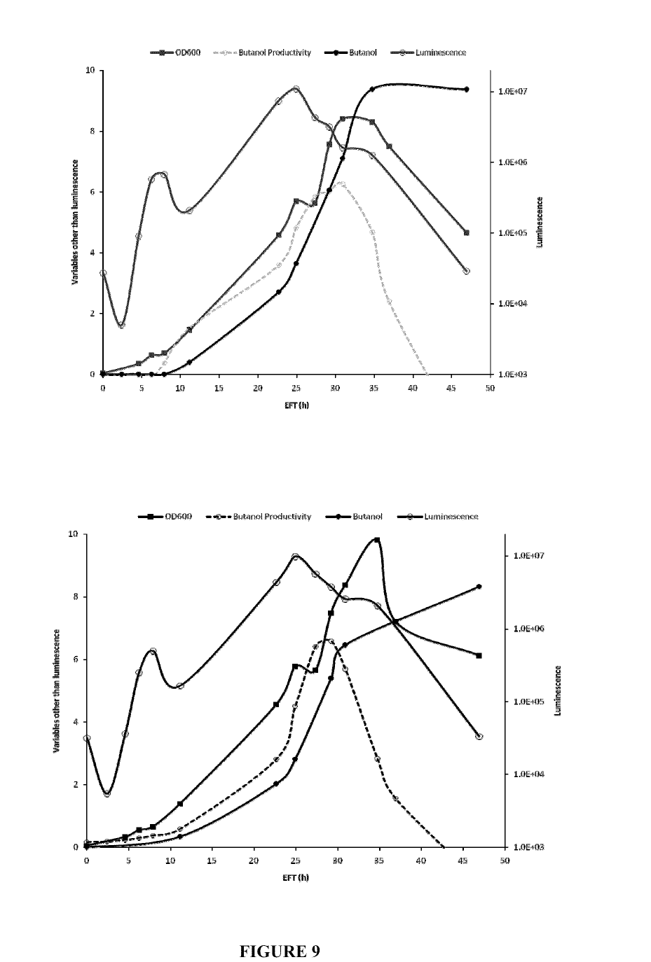

Further experiments confirmed the sensitivity of lux-based bioluminescence for detecting the production of a fermentation product. FIGS. 8 and 9. Bioluminescence was detectable hours earlier during batch culture than was butanol even though butanol detection relied on two very sensitive analytical methods, mass spectrometry of culture offgas and HPLC analysis of fermentation broth. FIG. 8. The reduction in bioluminescence also correlated with the ceasation of butanol production, unlike mass spectrometry or HPLC that can only measure the accumulation of a product.

The bioluminescence signal is reproducible as demonstrated in two different batch cultures. FIG. 9. Again, bioluminescence is detectable hours earlier than butanol and bioluminescence correlates well with overall butanol productivity.

### Continuous Culture

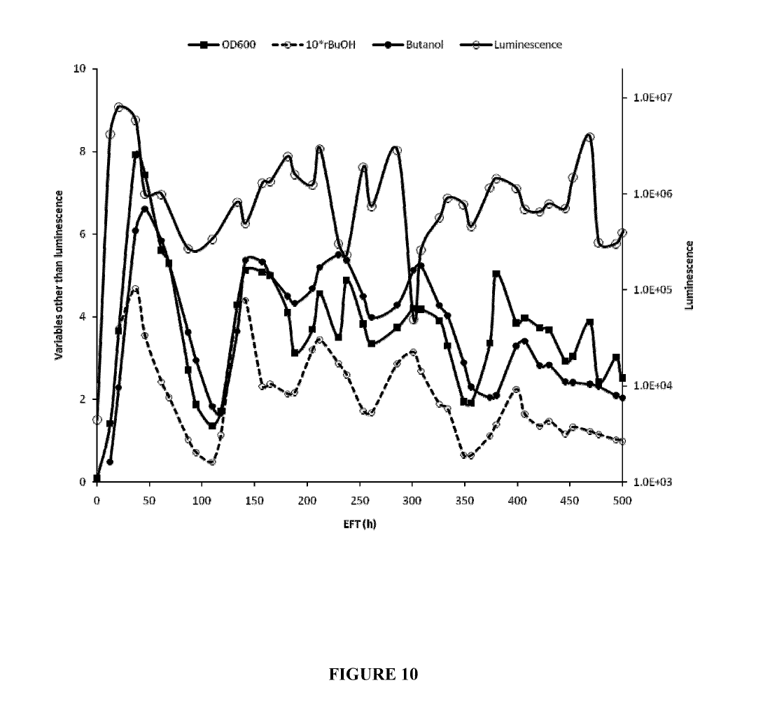

Bioluminescence in a continuous fermentation was also demonstrated to correlate with the fluctuations in the butanol production rate. FIG. 10.

### Use in Testing and Refining Culture Conditions

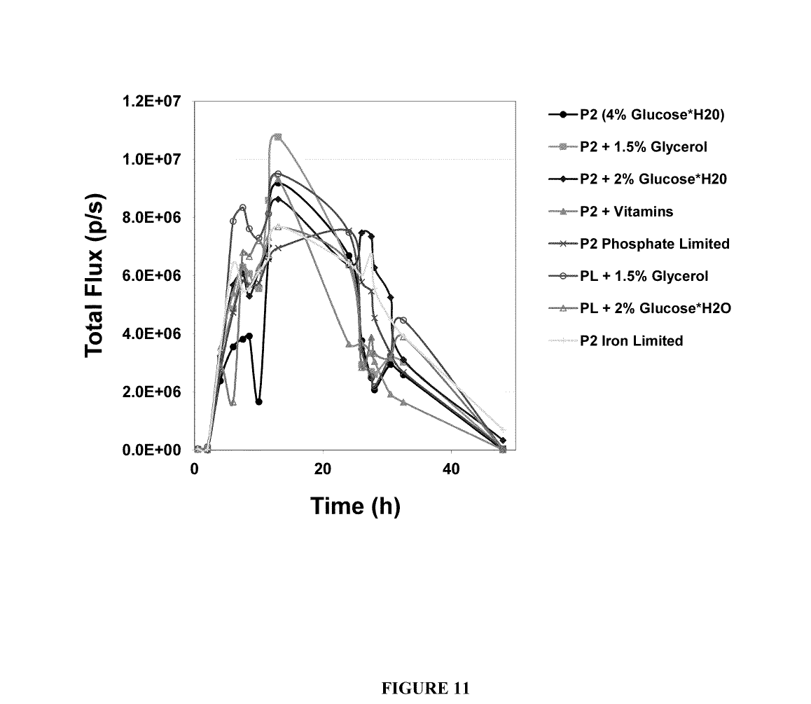

In addition to monitoring the production of a fermentative, metabolic or synthetic product, bioluminescence can be used to elucidate culture conditions that will reverse a decline, increase or maintain productivity of a given desired compound. FIG. 11 and Table 2. As the above examples demonstrate, the direct monitoring of an enzyme required for the production of product of interest through bioluminescence allows the detection of changes in the production rate hours before such change is evident from assaying the product in the offgas or fermentation broth. Valuable time can be saved by monitoring the metabolic flux of the microorganism directly through bioluminescence. FIG. 11 illustrates this utility, where eight different culture conditions were evaluated by bioluminescence using the C. beijerinckii strain Co-5878 (Co-0124 transformed with the inducible construct, pJIR418-bdhB::lux*). Two promising conditions identified by monitoring the metabolic flux, the addition of vitamins and phosphate limitation, were then tested in a 15 L fermentation runs. Table 2. Both conditions demonstrated higher butanol productivity compared to fermentations (n=3) conducted using the control media.

TABLE 2

| Batch | Yield | | | B:A ratio |
|---|---|---|---|---|
| Fermentation | g butanol/ | Titer | Pᵥ | g butanol/ |
| Performance | g glucose | g butanol/L | g butanol/L*h | g acetone |
| Co-5878 | 0.164 ± 0.001 | 10.7 ± 0.2 | 0.268 ± 0.005 | 2.83 ± 0.16 |
| Control Baseline | | | | |
| (n = 3) | | | | |
| Co-5878 | 0.167 | 10.6 | 0.299 | 2.95 |
| 2X Vitamins | | | | |
| (2008035) | | | | |
| Co-5878 | TBD | 9.9 | 0.282 | 1.65 |
| 0.1X [PO4] | | | | |
| (2008040) | | | | |

What is claimed is:

1. An isolated non-natural nucleic acid molecule comprising a nucleotide sequence encoding a light-emitting reporter, wherein the nucleotide sequence has an A/T content between about 62% and about 75%.

2. The nucleic acid molecule of claim 1 wherein the A/T content is between 65% and 75%.

3. The nucleic acid molecule of claim 1 wherein the light-emitting report is luciferase.

4. The nucleic acid molecule of claim 1 wherein the light-emitting report is self-contained.

5. The nucleic acid molecule of claim 1 wherein the nucleotide sequence encodes a Lux A polypeptide and/or a Lux B polypeptide.

6. The nucleic acid molecule of claim 1 wherein the nucleotide sequence encodes a Lux A polypeptide having the amino acid sequence of SEQ ID NO: 2.

7. The nucleic acid molecule of claim 6 wherein the nucleotide sequence comprises SEQ ID NO: 1.

8. The nucleic acid molecule of claim 1 wherein the nucleotide sequence encodes a Lux B polypeptide having the amino acid sequence of SEQ ID NO: 4.

9. The nucleic acid molecule of claim 8 wherein the nucleotide sequence comprises SEQ ID NO: 3.

10. The nucleic acid molecule of claim 1 wherein the nucleotide sequence is a polycistronic sequence.

11. The nucleic acid molecule of claim 10 wherein the polycistronic sequence encodes a lux A polypeptide and a lux B polypeptide.

12. The nucleic acid molecule of claim 11 wherein the lux A polypeptide and the lux B polypeptide are from Photorhabdus luminescens, or Vibrio fischeri.

13. The nucleic acid molecule of claim 11 wherein the lux A polypeptide and the lux B polypeptide are from a genus of organisms selected from a group consisting of Photorhabdus, Kenorhabdus, or Vibrio.

14. The nucleic acid molecule of claim 10 wherein the polycistronic sequence encodes a lux A polypeptide, a lux B polypeptide, a lux C polypeptide, a lux D polypeptide and a lux E polypeptide.

15. The nucleic acid molecule of claim 14 wherein the lux polypeptides are from Photorhabdus luminescens.

16. The nucleic acid molecule of claim 12 wherein the lux C polypeptide has the amino acid sequence of SEQ ID NO: 6, the lux D polypeptide has the amino acid sequence of SEQ ID NO: 5 and the lux E polypeptide has the amino acid sequence of SEQ ID NO: 10.

17. The nucleic acid molecule of claim 16 wherein the nucleotide sequence encoding the lux C polypeptide comprises SEQ ID NO: 6, the nucleotide sequence encoding the lux D polypeptide comprises SEQ ID NO: 8 and the nucleotide sequence encoding the lux E polypeptide comprises SEQ ID NO: 10.

18. The nucleic acid molecule of claim 1 wherein the nucleotide sequence encodes a lux A polypeptide and/or a lux B polypeptide having a non-natural amino acid sequence.

19. A recombinant nucleic acid molecule comprising an expression control sequence operatively linked with a coding nucleotide sequence encoding a light-emitting reporter, wherein the coding nucleotide sequence has an A/T content between about 62% and about 75%.

20. The recombinant nucleic acid molecule of claim 19 wherein the A/T content is between 65% and 75%.

21. The recombinant nucleic acid molecule of claim 19 wherein the light emitting report is luciferase.

22. The recombinant nucleic acid molecule of claim 19 which is a plasmid.

23. The recombinant nucleic acid molecule of claim 19 which is a transposon.

24. The recombinant nucleic acid molecule of claim 19 wherein the expression control sequence functions in a gram positive bacterium.

25. The recombinant nucleic acid molecule of claim 19 wherein the expression control sequence comprises a promoter that is functional in Clostridium.

26. The recombinant nucleic acid molecule of claim 24 wherein the promoter selected from the group consisting of promoters of genes for butanol dehydrogenase, butyraldehyde dehydrogenase, ethanol dehydrogenase, acid aldehyde dehydrogenase, acetoacetate decarboxylase, butyrate kinase, phosphobutyryltransferase, phosphotransacetylase, acetate kinase, acyl CoA transferase, lactate dehydrogenase and butryl CoA transferase.

27. The recombinant nucleic acid molecule of claim 19 comprising an operon wherein the coding nucleotide sequence is a polycistronic sequence.

28. The recombinant nucleic acid molecule of claim 27 wherein the polycistronic sequence encodes a lux A polypeptide and a lux B polypeptide.

29. The recombinant nucleic acid molecule of claim 28 wherein the polycistronic sequence further encodes a lux C polypeptide, a lux D polypeptide and a lux E polypeptide.

30. The recombinant nucleic acid molecule of claim 29 wherein the polycistronic sequence comprises SEQ ID NO: 12.

31. The recombinant nucleic acid molecule of claim 29 wherein the polycistronic sequence further encodes a lux R polypeptide and a lux I polypeptide.

32. The recombinant nucleic acid molecule of claim 27 wherein the expression control sequence comprises a Shine-Dalgarno sequence (AGGAGG) operatively linked with each cistron.

33. The recombinant nucleic acid molecule of claim 19 comprising a first restriction sequence upstream of the expression control sequence, a second restriction sequence between a promoter of the expression control sequence and the coding nucleotide sequence and a third restriction sequence downstream of the coding nucleotide sequence.

34. The recombinant nucleic acid molecule of claim 28 comprising a first restriction sequence and a second restriction sequence upstream and downstream, respectively, of the sequence encoding the lux A polypeptide and the lux B polypeptide.

35. A recombinant cell comprising a recombinant nucleic acid molecule comprising an expression control sequence operatively linked with a coding nucleotide sequence encoding a light-emitting reporter, wherein the coding nucleotide sequence has an A/T content between about 62% and about 75%.

36. The recombinant cell of claim 35 wherein the cell is a Clostridium cell.

37. The recombinant cell of claim 36 wherein Clostridium is C. acetobutylicum, C. perfringens, C. saccharobutylicum, C. puniceum, C. saccharoperobutylicum or C. beijerinckii.

38. The recombinant cell of claim 35 wherein the cell is and other bacteria with an AT rich DNA.

39. The recombinant cell of claim 35 wherein the recombinant nucleic acid is not integrated into the cell genome.

40. The recombinant cell of claim 35 wherein the recombinant nucleic acid is integrated into the cell genome.

41. The recombinant cell of claim 35 comprising a plurality of different recombinant nucleic acid molecules, wherein the different recombinant nucleic acid molecules comprise different expression control sequences and different coding nucleotide sequences encoding light-emitting reporters that report light of different wavelengths.

42. An isolated polypeptide comprising a light-emitting reporter, wherein the polypeptide is encoded by a nucleotide sequence having an A/T content from between about 62% and about 75%.

43. The polypeptide of claim 42 wherein the A/T content is between 65% and 75%.

44. The polypeptide of claim 42 wherein the light-emitting reporter is luciferase.

45. The polypeptide of claim 42 wherein the light-emitting report is self-contained.

46. The polypeptide of claim 42 further comprising a Lux A polypeptide and/or a Lux B polypeptide.

47. The polypeptide of claim 42 further having the amino acid sequence of SEQ ID NO: 2.

48. The polypeptide of claim 47 encoded by the nucleotide sequence of SEQ ID NO: 1.

49. The polypeptide of claim 42 further having having the amino acid sequence of SEQ ID NO: 4.

50. The polypeptide of claim 42 encoded by the nucleotide sequence comprises SEQ ID NO: 3.

51. The polypeptide of claim 42 wherein the nucleotide sequence is a polycistronic sequence encoding luciferase.

52. The polypeptide of claim 51 wherein the polycistronic sequence encodes a lux A polypeptide and a lux B polypeptide.

53. The polypeptide of claim 52 wherein the lux A polypeptide and the lux B polypeptide are from Photorhabdus luminescens, or Vibrio fischeri.

54. The polypeptide of claim 52 wherein the lux A polypeptide and the lux B polypeptide are from a genus of organisms selected from a group consisting of Photorhabdus, Kenorhabdus, and Vibrio.

55. The polypeptide of claim 51 wherein the polycistronic sequence encodes a lux A polypeptide, a lux B polypeptide, a lux C polypeptide, a lux D polypeptide and a lux E polypeptide.

56. The polypeptide of claim 55 wherein the lux polypeptides are from Photorhabdus luminescens.

57. A method comprising:
a) culturing a recombinant cell comprising a recombinant nucleic acid molecule comprising an expression control sequence operatively linked with a coding nucleotide sequence encoding a light-emitting reporter, wherein the coding nucleotide sequence has an A/T content between about 62% and about 75%; and
b) measuring the light emitted from the reporter in the culture.

58. The method of claim 57 wherein the light-emitting reporter is self-contained.

59. The method of claim 57 wherein the cell is Clostridium and the expression control sequence comprises a Clostridium promoter.

60. The method of claim 57 wherein the expression control sequence is from a low-GC bacteria.

61. The method of claim 60 wherein the light-emitting reporter is from Photorhabdus luminescens.

62. A method for regulating fermentation in a bacterial cell culture comprising; monitoring expression of a light emitting reporter in bacteria in said culture, wherein said light emitting reporter is encoded by a nucleic acid sequence comprising total A/T content of about 62% to about 75%; wherein said bacteria is low-GC bacteria; and regulating conditions in said culture based on said monitoring.

63. A method for identifying and/or optimizing fermentation culture conditions comprising: culturing a plurality of cultures, wherein the bacteria are the same, wherein the culture conditions are different, and wherein one culture condition serves as a control condition; monitoring the expression of a light emitting reporter in said bacteria in said cultures, wherein said light emitting reporter is encoded by a nucleic acid sequence comprising total A/T content of about 62% to about 75%; and wherein said bacteria is low-GC bacteria; and identifying said cultures that have a higher expression of the light emitting reporter compared to a control culture.

64. The method of claim 63, wherein said cultures with a higher expression of the light emitting report compared to the control culture indicate culture conditions that will result in higher productivity than a control culture condition.

65. The method of claim 63, wherein the culture conditions vary by nutrient, vitamin, mineral, salt, or cofactor composition.

66. The method of claim 63, wherein the culture conditions vary by a physical parameter selected from temperature, pH, oxygen partial pressure, osmotic pressure, or dilution rate of said culture.

67. A method for identifying mutants with higher productivity comprising: mutagenizing a plurality of bacteria that express a recombinant nucleic acid molecule comprising an expression control sequence operatively linked with a coding nucleotide sequence encoding a light-emitting reporter, wherein the coding nucleotide sequence has an A/T content between about 62% and about 75%; isolating pure cultures derived from individual mutants; culturing the pure cultures of mutants; measuring the light emitted from the reporter in the cultures; and selecting mutants that have a higher emission of light than an unmutagenized parent strain.

68. The method of claim 60, wherein said bacteria is Clostridium.

69. A kit comprising:
a) a first container containing a first nucleic acid molecule comprising an expression control sequence and
b) a second container containing a second nucleic acid molecule comprising a coding nucleotide sequence encoding a light-emitting reporter, wherein the coding nucleotide sequence has an A/T content between about 65% and about 75%;
c) wherein the first and second nucleic acid molecules comprise compatible restriction sequences which, when the first and second nucleic acid molecules are ligated together, put the expression control sequence in operative linkage with the coding nucleotide sequence and create a restriction sequence.

70. A kit containing:
a) a Clostridium cell; and
b) recombinant nucleic acid molecule comprising an expression control sequence operatively linked with a coding nucleotide sequence encoding a light-emitting reporter, wherein the coding nucleotide sequence has an A/T content between about 62% and about 75%.
